SEQUENCE LISTING (93 sequences)

SEQ ID NO: 1 - DNA, 11 bp, Artificial Sequence
  synthetic polynucleotide sequence
cacctcgtgac

SEQ ID NO: 2 - protein, 199 aa, Mus musculus
MSARAPKELRLALPPCLLNRTFASHNASGGSSAGLRSSGAGGGTCITQVGQQLFQSFSSTLVLIVLVTLIFCLLVLSLSTFHIHKRRMKKRKMQRAQEEYERDHCSGSHGGGGLPRAGVQAPTHGKETRLERQPRDSAFCTPSNATSSSSSSSSSPGLLCQGPCAPPPPLPAPTPQGAPAASSCLDTPGEGLLQTVVLS

SEQ ID NO: 3 - protein, 197 aa, Homo sapiens
MSARAPKELRLALPPCLLNRTFASPNASGSGNTGARGPGAVGSGTCITQVGQQLFQSFSSTLVLIVLVTLIFCLIVLSLSTFHIHKRRMKKRKMQRAQEEYERDHCSGSRGGGGLPRPGRQAPTHAKETRLERQPRDSPFCAPSNASSLSSSSPGLPCQGPCAPPPPPPASSPQGAHAASSCLDTAGEGLLQTVVLS

SEQ ID NO: 4 - protein, 619 aa, Homo sapiens
MSGIKKQKTENQQKSTNVVYQAHHVSRNKRGQVVGTRGGFRGCTVWLTGLSGAGKTTISFALEEYLVSHAIPCYSLDGDNVRHGLNRNLGFSPGDREENIRRIAEVAKLFADAGLVCITSFISPFAKDRENARKIHESAGLPFFEIFVDAPLNICESRDVKGLYKRARAGEIKGFTGIDSDYEKPETPERVLKTNLSTVSDCVHQVVELLQEQNIVPYTIIKDIHELFVPENKLDHVRAEAETLPSLSITKLDLQWVQVLSEGWATPLKGFMREKEYLQVMHFDTLLDGMALPDGVINMSIPIVLPVSAEDKTRLEGCSKFVLAHGGRRVAILRDAEFYEHRKEERCSRVWGTTCTKHPHIKMVMESGDWLVGGDLQVLEKIRWNDGLDQYRLTPLELKQKCKEMNADAVFAFQLRNPVHNGHALLMQDTRRRLLERGYKHPVLLLHPLGGWTKDDDVPLDWRMKQHAAVLEEGVLDPKSTIVAIFPSPMLYAGPTEVQWHCRSRMIAGANFYIVGRDPAGMPHPETKKDLYEPTHGGKVLSMAPGLTSVEIIPFRVAAYNKAKKAMDFYDPARHNEFDFISGTRMRKLAREGENPPDGFMAPKAWKVLTDYYRSLEKN

SEQ ID NO: 5 - protein, 382 aa, Homo sapiens
MRSPLCWLLPLLILASVAQGQPTRRPRPGTGPGRRPRPRPRPTPSFPQPDEPAEPTDLPPPLPPGPPSIFPDCPRECYCPPDFPSALYCDSRNLRKVPVIPPRIHYLYLQNNFITELPVESFQNATGLRWINLDNNRIRKIDQRVLEKLPGLVFLYMEKNQLEEVPSALPRNLEQLRLSQNHISRIPPGVFSKLENLLLLDLQHNRLSDGVFKPDTFHGLKNLMQLNLAHNILRKMPPRVPTAIHQLYLDSNKIETIPNGYFKSFPNLAFIRLNYNKLTDRGLPKNSFNISNLLVLHLSHNRISSVPAINNRLEHLYLNNNSIEKINGTQICPNDLVAFHDFSSDLENVPHLRYLRLDGNYLKPPIPLDLMMCFRLLQSVVI

SEQ ID NO: 6 - protein, 401 aa, Homo sapiens
MNNLLCCALVFLDISIKWTTQETFPPKYLHYDEETSHQLLCDKCPPGTYLKQHCTAKWKTVCAPCPDHYYTDSWHTSDECLYCSPVCKELQYVKQECNRTHNRVCECKEGRYLEIEFCLKHRSCPPGFGVVQAGTPERNTVCKRCPDGFFSNETSSKAPCRKHTNCSVFGLLLTQKGNATHDNICSGNSESTQKCGIDVTLCEEAFFRFAVPTKFTPNWLSVLVDNLPGTKVNAESVERIKRQHSSQEQTFQLLKLWKHQNKDQDIVKKIIQDIDLCENSVQRHIGHANLTFEQLRSLMESLPGKKVGAEDIEKTIKACKPSDQILKLLSLWRIKNGDQDTLKGLMHALKHSKTYHFPKTVTQSLKKTIRFLHSFTMYKLYQKLFLEMIGNQVQSVKISCL

SEQ ID NO: 7 - protein, 147 aa, Homo sapiens
MALQRTHSLLLLLLLTLLGLGLVQPSYGQDGMYQRFLRQHVHPEETGGSDRYCNLMMQRRKMTLYHCKRFNTFIHEDIWNIRSICSTTNIQCKNGKMNCHEGVVKVTDCRDTGSSRAPNCRYRAIASTRRVVIACEGNPQVPVHFDG

SEQ ID NO: 8 - protein, 1211 aa, Homo sapiens
MDPPAGAARRLLCPALLLLLLLLPPPLLPPPPPPANARLAAAADPPGGPLGHGAERILAVPVRTDAQGRLVSHVVSAATSRAGVRARRAAPVRTPSFPGGNEEEPGSHLFYNVTVFGRDLHLRLRPNARLVAPGATMEWQGEKGTTRVEPLLGSCLYVGDVAGLAEASSVALSNCDGLAGLIRMEEEEFFIEPLEKGLAAQEAEQGRVHVVYRRPPTSPPLGGPQALDTGASLDSLDSLSRALGVLEEHANSSRRRARRHAADDDYNIEVLLGVDDSVVQFHGKEHVQKYLLTLMNIVNEIYHDESLGAHINVVLVRIILLSYGKSMSLIEIGNPSQSLENVCRWAYLQQKPDTGHDEYHDHAIFLTRQDFGPSGMQGYAPVTGMCHPVRSCTLNHEDGFSSAFVVAHETGHVLGMEHDGQGNRCGDEVRLGSIMAPLVQAAFHRFHWSRCSQQELSRYLHSYDCLLDDPFAHDWPALPQLPGLHYSMNEQCRFDFGLGYMMCTAFRTFDPCKQLWCSHPDNPYFCKTKKGPPLDGTMCAPGKHCFKGHCIWLTPDILKRDGSWGAWSPFGSCSRTCGTGVKFRTRQCDNPHPANGGRTCSGLAYDFQLCSRQDCPDSLADFREEQCRQWDLYFEHGDAQHHWLPHEHRDAKERCHLYCESRETGEVVSMKRMVHDGTRCSYKDAFSLCVRGDCRKVGCDGVIGSSKQEDKCGVCGGDNSHCKVVKGTFTRSPKKHGYIKMFEIPAGARHLLIQEVDATSHHLAVKNLETGKFILNEENDVDASSKTFIAMGVEWEYRDEDGRETLQTMGPLHGTITVLVIPVGDTRVSLTYKYMIHEDSLNVDDNNVLEEDSVVYEWALKKWSPCSKPCGGGSQFTKYGCRRRLDHKMVHRGFCAALSKPKAIRRACNPQECSQPVWVTGEWEPCSQTCGRTGMQVRSVRCIQPLHDNTTRSVHAKHCNDARPESRRACSRELCPGRWRAGPWSQCSVTCGNGTQERPVLCRTADDSFGICQEERPETARTCRLGPCPRNISDPSKKSYVVQWLSRPDPDSPIRKISSKGHCQGDKSIFCRMEVLSRYCSIPGYNKLCCKSCNLYNNLTNVEGRIEPPPGKHNDIDVFMPTLPVPTVAMEVRPSPSTPLEVPLNASSTNATEDHPETNAVDEPYKIHGLEDEVQPPNLIPRRPSPYEKTRNQRIQELIDEMRKKEMLGKF

SEQ ID NO: 9 - protein, 591 aa, Homo sapiens
MAPEINLPGPMSLIDNTKGQLVVNPEALKILSAITQPVVVVAIVGLYRTGKSYLMNKLAGKKNGFSLGSTVKSHTKGIWMWCVPHPKKPEHTLVLLDTEGLGDIEKGDNENDSWIFALAILLSSTFVYNSMGTINQQAMDQLHYVTELTDRIKANSSPGNNSVDDSADFVSFFPAFVWTLRDFTLELEVDGEPITADDYLELSLKLRKGTDKKSKSFNDPRLCIRKFFPKRKCFVFDWPAPKKYLAHLEQLKEEELNPDFIEQVAEFCSYILSHSNVKTLSGGIPVNGPRLESLVLTYVNAISSGDLPCMENAVLALAQIENSAAVEKAIAHYEQQMGQKVQLPTETLQELLDLHRDSEREAIEVFMKNSFKDVDQMFQRKLGAQLEARRDDFCKQNSKASSDCCMALLQDIFGPLEEDVKQGTFSKPGGYRLFTQKLQELKNKYYQVPRKGIQAKEVLKKYLESKEDVADALLQTDQSLSEKEKAIEVERIKAESAEAAKKMLEEIQKKNEEMMEQKEKSYQEHVKQLTEKMERDRAQLMAEQEKTLALKLQEQERLLKEGFENESKRLQKDIWDIQMRSKSLEPICNIL

SEQ ID NO: 10 - protein, 98 aa, Homo sapiens
MAAEPLTELEESIETVVTTFFTFARQEGRKDSLSVNEFKELVTQQLPHLLKDVGSLDEKMKSLDVNQDSELKFNEYWRLIGELAKEIRKKKDLKIRKK

SEQ ID NO: 11 - protein, 379 aa, Homo sapiens
MVWVVLLSLLCYLVLFLCRHSSHRGVFLSVTILIYLLMGEMHMVDTVTWHKMRGAQMIVAMKAVSLGFDLDRGEVGTVPSPVEFMGYLYFVGTIVFGPWISFHSYLQAVQGRPLSCRWLQKVARSLALALLCLVLSTCVGPYLFPYFIPLNGDRLLRKWLRAYESAVSFHFSNYFVGFLSEATATLAGAGFTEEKDHLEWDLTVSKPLNVELPRSMVEVVTSWNLPMSYWLNNYVFKNALRLGTFSAVLVTYAASALLHGFSFHLAAVLLSLAFITYVEHVLRKRLARILSACVLSKRCPPDCSHQHRLGLGVRALNLLFGALAIFHLAYLGSLFDVDVDDTTEEQGYGMAYTVHKWSELSWASHWVTFGCWIFYRLIG

SEQ ID NO: 12 - protein, 403 aa, Homo sapiens
MYREQMNLTSLDPPLQLRLEASWVQFHLGINRHGLYSRSSPVVSKLLQDMRHFPTISADYSQDEKALLGACDCTQIVKPSGVHLKLVLRFSDFGKAMFKPMRQQRDEETPVDFFYFIDFQRHNAEIAAFHLDRILDFRRVPPTVGRIVNVTKEILEVTKNEILQSVFFVSPASNVCFFAKCPYMCKTEYAVCGNPHLLEGSLSAFLPSLNLAPRLSVPNPWIRSYTLAGKEEWEVNPLYCDTVKQIYPYNNSQRLLNVIDMAIFDFLIGNMDRHHYEMFTKFGDDGFLIHLDNARGFGRHSHDEISILSPLSQCCMIKKKTLLHLQLLAQADYRLSDVMRESLLEDQLSPVLTEPHLLALDRRLQTILRTVEGCIVAHGQQSVIVDGPVEQLAPDSGQANLTS

SEQ ID NO: 13 - protein, 333 aa, Homo sapiens
MCEEETTALVCDNGSGLCKAGFAGDDAPRAVFPSIVGRPRHQIWHHSFYNELRVAPEEHPTLLTEAPLNPKANREKMTQIMFETFNVPAMYVAIQAVLSLYASGRTTGIVLDSGDGVTHNVPIYEGYALPHAIMRLDLAGRDLTDYLMKILTERGYSFVTTAEREIVRDIKEKLCYVALDFENEMATAASSSSLEKSYELPDGQVITIGNERFRCPETLFQPSFIGMESAGIHETTYNSIMKCDIDIRKDLYANNVLSGGTTMYPGIADRMQKEITALAPSTMKIKIIAPPERKYSVWIGGSILASLSTFQQMWISKPEYDEAGPSIVHRKCF

SEQ ID NO: 14 - protein, 744 aa, Homo sapiens
MAVLPGPLQLLGVLLTISLSSIRLIQAGAYYGIKPLPPQIPPQMPPQIPQYQPLGQQVPHMPLAKDGLAMGKEMPHLQYGKEYPHLPQYMKEIQPAPRMGKEAVPKKGKEIPLASLRGEQGPRGEPGPRGPPGPPGLPGHGIPGIKGKPGPQGYPGVGKPGMPGMPGKPGAMGMPGAKGEIGQKGEIGPMGIPGPQGPPGPHGLPGIGKPGGPGLPGQPGPKGDRGPKGLPGPQGLRGPKGDKGFGMPGAPGVKGPPGMHGPPGPVGLPGVGKPGVTGFPGPQGPLGKPGAPGEPGPQGPIGVPGVQGPPGIPGIGKPGQDGIPGQPGFPGGKGEQGLPGLPGPPGLPGIGKPGFPGPKGDRGMGGVPGALGPRGEKGPIGAPGIGGPPGEPGLPGIPGPMGPPGAIGFPGPKGEGGIVGPQGPPGPKGEPGLQGFPGKPGFLGEVGPPGMRGLPGPIGPKGEAGQKGVPGLPGVPGLLGPKGEPGIPGDQGLQGPPGIPGIGGPSGPIGPPGIPGPKGEPGLPGPPGFPGIGKPGVAGLHGPPGKPGALGPQGQPGLPGPPGPPGPPGPPAVMPPTPPPQGEYLPDMGLGIDGVKPPHAYGAKKGKNGGPAYEMPAFTAELTAPFPPVGAPVKFNKLLYNGRQNYNPQTGIFTCEVPGVYYFAYHVHCKGGNVWVALFKNNEPVMYTYDEYKKGFLDQASGSAVLLLRPGDRVFLQMPSEQAAGLYAGQYVHSSFSGYLLYPM

SEQ ID NO: 15 - protein, 95 aa, Mus musculus
MRTLSLLTLLALAALCLSDLTDATPTGPESDKAFMSKQEGNKVVNRLRRYLGASVPSPDPLEPTRELCELDPACDELSNQYGLKTAYRRIYGITI

SEQ ID NO: 16 - protein, 103 aa, Homo sapiens
MKSLILLAILAALAVVTLCYESHESMESYELNPFINRRNANTFISPQQRWRAKVQERIRERSKPVHELNREACDDYRLCERYAMVYGYNAAYNRYFRKRRGTK

SEQ ID NO: 17 - protein, 471 aa, Homo sapiens
MHPGVLAAFLFLSWTHCRALPLPSGGDEDDLSEEDLQFAERYLRSYYHPTNLAGILKENAASSMTERLREMQSFFGLEVTGKLDDNTLDVMKKPRCGVPDVGEYNVFPRTLKWSKMNLTYRIVNYTPDMTHSEVEKAFKKAFKVWSDVTPLNFTRLHDGIADIMISFGIKEHGDFYPFDGPSGLLAHAFPPGPNYGGDAHFDDDETWTSSSKGYNLFLVAAHEFGHSLGLDHSKDPGALMFPIYTYTGKSHFMLPDDDVQGIQSLYGPGDEDPNPKHPKTPDKCDPSLSLDAITSLRGETMIFKDRFFWRLHPQQVDAELFLTKSFWPELPNRIDAAYEHPSHDLIFIFRGRKFWALNGYDILEGYPKKISELGLPKEVKKISAAVHFEDTGKTLLFSGNQVWRYDDTNHIMDKDYPRLIEEDFPGIGDKVDAVYEKNGYIYFFNGPIQFEYSIWSNRIVRVMPANSILWC

SEQ ID NO: 18 - protein, 1170 aa, Homo sapiens
MGLAWGLGVLFLMHVCGTNRIPESGGDNSVFDIFELTGAARKGSGRRLVKGPDPSSPAFRIEDANLIPPVPDDKFQDLVDAVRAEKGFLLLASLRQMKKTRGTLLALERKDHSGQVFSVVSNGKAGTLDLSLTVQGKQHVVSVEEALLATGQWKSITLFVQEDRAQLYIDCEKMENAELDVPIQSVFTRDLASIARLRIAKGGVNDNFQGVLQNVRFVFGTTPEDILRNKGCSSSTSVLLTLDNNVVNGSSPAIRTNYIGHKTKDLQAICGISCDELSSMVLELRGLRTIVTTLQDSIRKVTEENKELANELRRPPLCYHNGVQYRNNEEWTVDSCTECHCQNSVTICKKVSCPIMPCSNATVPDGECCPRCWPSDSADDGWSPWSEWTSCSTSCGNGIQQRGRSCDSLNNRCEGSSVQTRTCHIQECDKRFKQDGGWSHWSPWSSCSVTCGDGVITRIRLCNSPSPQMNGKPCEGEARETKACKKDACPINGGWGPWSPWDICSVTCGGGVQKRSRLCNNPTPQFGGKDCVGDVTENQICNKQDCPIDGCLSNPCFAGVKCTSYPDGSWKCGACPPGYSGNGIQCTDVDECKEVPDACFNHNGEHRCENTDPGYNCLPCPPRFTGSQPFGQGVEHATANKQVCKPRNPCTDGTHDCNKNAKCNYLGHYSDPMYRCECKPGYAGNGIICGEDTDLDGWPNENLVCVANATYHCKKDNCPNLPNSGQEDYDKDGIGDACDDDDDNDKIPDDRDNCPFHYNPAQYDYDRDDVGDRCDNCPYNHNPDQADTDNNGEGDACAADIDGDGILNERDNCQYVYNVDQRDTDMDGVGDQCDNCPLEHNPDQLDSDSDRIGDTCDNNQDIDEDGHQNNLDNCPYVPNANQADHDKDGKGDACDHDDDNDGIPDDKDNCRLVPNPDQKDSDGDGRGDACKDDFDHDSVPDIDDICPENVDISETDFRRFQMIPLDPKGTSQNDPNWVVRHQGKELVQTVNCDPGLAVGYDEFNAVDFSGTFFINTERDDDYAGFVFGYQSSSRFYVVMWKQVTQSYWDTNPTRAQGYSGLSVKVVNSTTGPGEHLRNALWHTGNTPGQVRTLWHDPRHIGWKDFTAYRWRLSHRPKTGFIRVVMYEGKKIMADSGPIYDKTYAGGRLGLFVFSQEMVFFSDLKYECRDP

SEQ ID NO: 19 - protein, 95 aa, Mus musculus
MRTIFLLTLLTLAALCLSDLTDAKPSGPESDKAFMSKQEGNKVVNRLRRYLGASVPSPDPLEPTREQCELNPACDELSDQYGLKTAYKRIYGITI

SEQ ID NO: 20 - protein, 100 aa, Homo sapiens
MRALTLLALLALAALCIAGQAGAKPSGAESSKGAAFVSKQEGSEVVKRPRRYLYQWLGAPVPYPDPLEPRREVCELNPDCDELADHIGFQEAYRRFYGPV

SEQ ID NO: 21 - protein, 95 aa, Mus musculus
MRTLSLLTLLALAALCLSDLTDAKPSGPESDKAFMSKQEGNKVVNRLRRYLGASVPSPDPLEPTREQCELNPACDELSDQYGLKTAYKRIYGITI

SEQ ID NO: 22 - protein, 377 aa, Homo sapiens
MCEEEDSTALVCDNGSGLCKAGFAGDDAPRAVFPSIVGRPRHQGVMVGMGQKDSYVGDEAQSKRGILTLKYPIEHGIITNWDDMEKIWHHSFYNELRVAPEEHPTLLTEAPLNPKANREKMTQIMFETFNVPAMYVAIQAVLSLYASGRTTGIVLDSGDGVTHNVPIYEGYALPHAIMRLDLAGRDLTDYLMKILTERGYSFVTTAEREIVRDIKEKLCYVALDFENEMATAASSSSLEKSYELPDGQVITIGNERFRCPETLFQPSFIGMESAGIHETTYNSIMKCDIDIRKDLYANNVLSGGTTMYPGIADRMQKEITALAPSTMKIKIIAPPERKYSVWIGGSILASLSTFQQMWISKQEYDEAGPSIVHRKCF

SEQ ID NO: 23 - protein, 44 aa, Homo sapiens
MSDKPDMAEIEKFDKSKLKKTETQEKNPLPSKETIEQEKQAGES

SEQ ID NO: 24 - protein, 373 aa, Homo sapiens
MRLLAWLIFLANWGGARAEPGKFWHIADLHLDPDYKVSKDPFQVCPSAGSQPVPDAGPWGDYLCDSPWALINSSIYAMKEIEPEPDFILWTGDDTPHVPDEKLGEAAVLEIVERLTKLIREVFPDTKVYAALGNHDFHPKNQFPAGSNNIYNQIAELWKPWLSNESIALFKKGAFYCEKLPGPSGAGRIVVLNTNLYYTSNALTADMADPGQQFQWLEDVLTDASKAGDMVYIVGHVPPGFFEKTQNKAWFREGFNEKYLKVVRKHHRVIAGQFFGHHHTDSFRMLYDDAGVPISAMFITPGVTPWKTTLPGVVNGANNPAIRVFEYDRATLSLKVRSPAEARGGGWEGLKCITTFPHSQLIHLPLTTEPQEG

SEQ ID NO: 25 - protein, 323 aa, Homo sapiens
MSRPRMRLVVTADDFGYCPRRDEGIVEAFLAGAVTSVSLLVNGAATESAAELARRHSIPTGLHANLSEGRPVGPARRGASSLLGPEGFFLGKMGFREAVAAGDVDLPQVREELEAQLSCFRELLGRAPTHADGHQHVHVLPGVCQVFAEALQAYGVRFTRLPLERGVGGCTWLEAPARAFACAVERDARAAVGPFSRHGLRWTDAFVGLSTCGRHMSAHRVSGALARVLEGTLAGHTLTAELMAHPGYPSVPPTGGCGEGPDAFSCSWERLHELRVLTAPTLRAQLAQDGVQLCALDDLDSKRPGEEVPCEPTLEPFLEPSLL

SEQ ID NO: 26 - protein, 345 aa, Homo sapiens
MGLDLPDGGHLTHGFMTDKKKISATSIFFESMPYKVNPDTGYINYDQLEENARLFHPKLIIAGTSCYSRNLEYARLRKIADENGAYLMADMAHISGLVAAGVVPSPFEHCHVVTTTTHKTLRGCRAGMIFYRKGVKSVDPKTGKEILYNLESLINSAVFPGLQGGPHNHAIAGVAVALKQAMTLEFKVYQHQVVANCRALSEALTELGYKIVTGGSDNHLILVDLRSKGTDGGRAEKVLEACSIACNKNTCPGDRSALRPSGLRLGTPALTSRGLLEKDFQKVAHFIHRGIELTLQIQSDTGVRATLKEFKERLAGDKYQAAVQALREEVESFASLFPLPGLPDF

SEQ ID NO: 27 - protein, 475 aa, Homo sapiens
MESKALLVLTLAVWLQSLTASRGGVAAADQRRDFIDIESKFALRTPEDTAEDTCHLIPGVAESVATCHFNHSSKTFMVIHGWTVTGMYESWVPKLVAALYKREPDSNVIVVDWLSRAQEHYPVSAGYTKLVGQDVARFINWMEEEFNYPLDNVHLLGYSLGAHAAGIAGSLTNKKVNRITGLDPAGPNFEYAEAPSRLSPDDADFVDVLHTFTRGSPGRSIGIQKPVGHVDIYPNGGTFQPGCNIGEAIRVIAERGLGDVDQLVKCSHERSIHLFIDSLLNEENPSKAYRCSSKEAFEKGLCLSCRKNRCNNLGYEINKVRAKRSSKMYLKTRSQMPYKVFHYQVKIHFSGTESETHTNQAFEISLYGTVAESENIPFTLPEVSTNKTYSFLIYTEVDIGELLMLKLKWKSDSYFSWSDWWSSPGFAIQKIRVKAGETQKKVIFCSREKVSHLQKGKAPAVFVKCHDKSLNKKSG

SEQ ID NO: 28 - protein, 1887 aa, Homo sapiens
MAARGRGLLLLTLSVLLAAGPSAAAAKLNIPKVLLPFTRATRVNFTLEASEGCYRWLSTRPEVASIEPLGLDEQQCSQKAVVQARLTQPARLTSIIFAEDITTGQVLRCDAIVDLIHDIQIVSTTRELYLEDSPLELKIQALDSEGNTFSTLAGLVFEWTIVKDSEADRFSDSHNALRILTFLESTYIPPSYISEMEKAAKQGDTILVSGMKTGSSKLKARIQEAVYKNVRPAEVRLLILENILLNPAYDVYLMVGTSIHYKVQKIRQGKITELSMPSDQYELQLQNSIPGPEGDPARPVAVLAQDTSMVTALQLGQSSLVLGHRSIRMQGASRLPNSTIYVVEPGYLGFTVHPGDRWVLETGRLYEITIEVFDKFSNKVYVSDNIRIETVLPAEFFEVLSSSQNGSYHRIRALKRGQTAIDAALTSVVDQDGGVHILQVPVWNQQEVEIHIPITLYPSILTFPWQPKTGAYQYTIRAHGGSGNFSWSSSSHLVATVTVKGVMTTGSDIGFSVIQAHDVQNPLHFGEMKVYVIEPHSMEFAPCQVEARVGQALELPLRISGLMPGGASEVVTLSDCSHFDLAVEVENQGVFQPLPGRLPPGSEHCSGIRVKAEAQGSTTLLVSYRHGHVHLSAKITIAAYLPLKAVDPSSVALVTLGSSKEMLFEGGPRPWILEPSKFFQNVTAEDTDSIGLALFAPHSSRNYQQHWILVTCQALGEQVIALSVGNKPSLTNPFPAVEPAVVKFVCAPPSRLTLAPVYTSPQLDMSCPLLQQNKQVVPVSSHRNPRLDLAAYDQEGRRFDNFSSLSIQWESTRPVLASIEPELPMQLVSQDDESGQKKLHGLQAILVHEASGTTAITATATGYQESHLSSARTKQPHDPLVPLSASIELILVEDVRVSPEEVTIYNHPGIQAELRIREGSGYFFLNTSTADVVKVAYQEARGVAMVHPLLPGSSTIMIHDLCLVFPAPAKAVVYVSDIQELYIRVVDKVEIGKTVKAYVRVLDLHKKPFLAKYFPFMDLKLRAASPIITLVALDEALDNYTITFLIRGVAIGQTSLTASVTNKAGQRINSAPQQIEVFPPFRLMPRKVTLLIGATMQVTSEGGPQPQSNILFSISNESVALVSAAGLVQGLAIGNGTVSGLVQAVDAETGKVVIISQDLVQVEVLLLRAVRIRAPIMRMRTGTQMPIYVTGITNHQNPFSFGNAVPGLTFHWSVTKRDVLDLRGRHHEASIRLPSQYNFAMNVLGRVKGRTGLRVVVKAVDPTSGQLYGLARELSDEIQVQVFEKLQLLNPEIEAEQILMSPNSYIKLQTNRDGAASLSYRVLDGPEKVPVVHVDEKGFLASGSMIGTSTIEVIAQEPFGANQTIIVAVKVSPVSYLRVSMSPVLHTQNKEALVAVPLGMTVTFTVHFHDNSGDVFHAHSSVLNFATNRDDFVQIGKGPTNNTCVVRTVSVGLTLLRVWDAEHPGLSDFMPLPVLQAISPELSGAMVVGDVLCLATVLTSLEGLSGTWSSSANSILHIDPKTGVAVARAVGSVTVYYEVAGHLRTYKEVVVSVPQRIMARHLHPIQTSFQEATASKVIVAVGDRSSNLRGECTPTQREVIQALHPETLISCQSQFKPAVFDFPSQDVFTVEPQFDTALGQYFCSITMHRLTDKQRKHLSMKKTALVVSASLSSSHFSTEQVGAEVPFSPGLFADQAEILLSNHYTSSEIRVFGAPEVLENLEVKSGSPAVLAFAKEKSFGWPSFITYTVGVLDPAAGSQGPLSTTLTFSSPVTNQAIAIPVTVAFVVDRRGPGPYGASLFQHFLDSYQVMFFTLFALLAGTAVMIIAYHTVCTPRDLAVPAALTPRASPGHSPHYFAASSPTSPNALPPARKASPPSGLWSPAYASH

SEQ ID NO: 29 - protein, 246 aa, Homo sapiens
MPHSVTLRGPSPWGFRLVGGRDFSAPLTISRVHAGSKAALAALCPGDLIQAINGESTELMTHLEAQNRIKGCHDHLTLSVSRPEGRSWPSAPDDSKAQAHRIHIDPEIQDGSPTTSRRPSGTGTGPEDGRPSLGSPYGQPPRFPVPHNGSSEATLPAQMSTLHVSPPPSADPARGLPRSRDCRVDLGSEVYRMLREPAEPVAAEPKQSGSFRYLQGMLEAGEGGAPSSRHGTSSTIPSASCAVTAA

SEQ ID NO: 30 - protein, 262 aa, Homo sapiens
MDPRLSTVRQTCCCFNVRIATTALAIYHVIMSVLLFIEHSVEVAHGKASCKLSQMGYLRIADLISSFLLITMLFIISLSLLIGVVKNREKYLLPFLSLQIMDYLLCLLTLLGSYIELPAYLKLASRSRASSSKFPLMTLQLLDFCLSILTLCSSYMEVPTYLNFKSMNHMNYLPSQEDMPHNQFIKMMIIFSIAFITVLIFKVYMFKCVWRCYRLIKCMNSVEEKRNSKMLQKVVLPSYEEALSLPSKTPEGGPAPPPYSEV

SEQ ID NO: 31 - protein, 676 aa, Homo sapiens
MSRRTRCEDLDELHYQDTDSDVPEQRDSKCKVKWTHEENRTDQQCQYRWLRVLNPDLVKGPWTKEEDQKVIELVKKYGTKQWTLIAKHLKGRLGKQCRERWHNHLNPEVKKSCWTEEEDRIICEAHKVLGNRWAEIAKMLPGRTDNAVKNHWNSTIKRKVDTGGFLSESKDCKPPVYLLLELEDKDGLQSAQPTEGQGSLLTNWPSVPPTIKEEENSEEELAAATTSKEQEPIGTDLDAVRTPEPLEEFPKREDQEGSPPETSLPYKWVVEAANLLIPAVGSSLSEALDLIESDPDAWCDLSKFDLPEEPSAEDSINNSLVQLQASHQQQVLPPRQPSALVPSVTEYRLDGHTISDLSRSSRGELIPISPSTEVGGSGIGTPPSVLKRQRKRRVALSPVTENSTSLSFLDSCNSLTPKSTPVKTLPFSPSQFLNFWNKQDTLELESPSLTSTPVCSQKVVVTTPLHRDKTPLHQKHAAFVTPDQKYSMDNTPHTPTPFKNALEKYGPLKPLPQTPHLEEDLKEVLRSEAGIELIIEDDIRPEKQKRKPGLRRSPIKKVRKSLALDIVDEDVKLMMSTLPKSLSLPTTAPSNSSSLTLSGIKEDNSLLNQGFLQAKPEKAAVAQKPRSHFTTPAPMSSAWKTVACGGTRDQLFMQEKARQLLGRLKPSHTSRTLILS

SEQ ID NO: 32 - protein, 198 aa, Homo sapiens
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGLKGKVYQEPLSPNACMDTTAILQPHRSCLTHGS

SEQ ID NO: 33 - protein, 177 aa, Mus musculus
MPTGRTDGRTHGRTPQGRKPAPTAPLHPPQHTGHTRAPRPPRHTRHTRHTRQAGQAHASAGPAAPATQTRTSRRGQDVHPPRSRCMCHRPSPRWTDGRTHARRQRPPVTAAAHSDVTHESTHVEADAVVKMSLPSPQDGRTDSTRCACRRGRQDGAILTEEGARQQGLTAYRNAPPQ

SEQ ID NO: 34 - protein, 504 aa, Homo sapiens
MEAAAQFFVESPDVVYGPEAIEAQYEYRTTRVSREGGVLKEANYYGSLTQAGTVSLGLDAEGQEVFVPFSAVLPMVAPNDLVFDGWDISSLNLAEAMRRAKVLDWGLQEQLWPHMEALRPRPSVYIPEFIAANQSARADNLIPGSRAQQLEQIRRDIRDFRSSAGLDKVIVLWTANTERFCEVIPGLNDTAENLLRTIELGLEVSPSTLFAVASILEGCAFLNGSPQNTLVPGALELAWQHRVFVGGDDFKSGQTKVKSVLVDFLIGSGLKTMSIVSYNHLGNNDGENLSAPLQFRSKEVSKSNVVDDMVQSNPVLYTPGEEPDHCVVIKYVPYVGDSKRALDEYTSELMLGGTNTLVLHNTCEDSLLAAPIMLDLALLTELCQRVSFCTDMDPEPQTFHPVLSLLSFLFKAPLVPPGSPVVNALFRQRSCIENILRACVGLPPQNHMLLEHKMERPGPSLKRVGPVAATYPMLNKKGPVPAATNGCTGDANGHLQEEPPMPTT

SEQ ID NO: 35 - protein, 354 aa, Homo sapiens
MPRRSLHAAAVLLLVILKEQPSSPAPVNGSKWTYFGPDGENSWSKKYPSCGGLLQSPIDLHSDILQYDASLTPLEFQGYNLSANKQFLLTNNGHSVKLNLPSDMHIQGLQSRYSATQLHLHWGNPNDPHGSEHTVSGQHFAAELHIVHYNSDLYPDASTASNKSEGLAVLAVLIEMGSFNPSYDKIFSHLQHVKYKGQEAFVPGFNIEELLPERTAEYYRYRGSLTTPPCNPTVLWTVFRNPVQISQEQLLALETALYCTHMDDPSPREMINNFRQVQKFDERLVYTSFSQVQVCTAAGLSLGIILSLALAGILGICIVVVVSIWLFRRKSIKKGDNKGVIYKPATKMETEAHA

SEQ ID NO: 36 - protein, 161 aa, Homo sapiens
MNLAISIALLLTVLQVSRGQKVTSLTACLVDQSLRLDCRHENTSSSPIQYEFSLTRETKKHVLFGTVGVPEHTYRSRTNFTSKYNMKVLYLSAFTSKDEGTYTCALHHSGHSPPISSQNVTVLRDKLVKCEGISLLAQNTSWLLLLLLSLSLLQATDFMSL

SEQ ID NO: 37 - protein, 276 aa, Mus musculus
MSLCEDMLLCNYRKCRIKLSGYAWVTACSHIFCDQHGSGEFSRSPAICPACNSTLSGKLDIVRTELSPSEEYKAMVLAGLRPEVVLDISSRALAFWTYQVHQERLYQEYNFSKAENHLKQMEKMYMQQIQSKNIELTSMKGEVISMKKVLEEYKKKFSDISEKLMERNRQYQKLQGLYDSLRLRNITIASQEGSLEPGMIPQSGVFGFPPGNNSKFSLDHIPVGNQGGGDEDVQFRPFFVCSPTAPEPINNFFSFASPSHEAEQQVCSRAFKAKRI

SEQ ID NO: 38 - protein, 766 aa, Homo sapiens
MKTPWKVLLGLLGAAALVTIITVPVVLLNKGTDDATADSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWSYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLP

SEQ ID NO: 39 - protein, 288 aa, Homo sapiens
MEYFGTISIGSPPQNFTVIFDTGSSNLWVPSVYCTSPACKTHSRFQPSQSSTYSQPGQSFSIQYGTGSLSGIIGADQVSVEGLTVVGQQFGESVTEPGQTFVDAEFDGILGLGYPSLAVGGVTPVFDNMMAQNLVDLPMFSVYMSSNPEGGAGSELIFGGYDHSHFSGSLNWVPVTKQAYWQIALDNMLWSVPTLTSCRMSPSPLTESPIPSAQLPTPYWTSWMECSSAAVAFKDLTSTLQLGPSGSWGMSSFDSFTQSLTVGITVWDWPQQSPKEGPCVCACLSDRP

SEQ ID NO: 40 - protein, 182 aa, Homo sapiens
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 41 - protein, 199 aa, Mus musculus; identical to SEQ ID NO: 2

SEQ ID NO: 42 - protein, 197 aa, Homo sapiens; identical to SEQ ID NO: 3

SEQ ID NO: 43 - protein, 614 aa, Homo sapiens
MSGIKKQKTENQQKSTNVVYQAHHVSRNKRGQVVGTRGGFRGCTVWLTGLSGAGKTTISFALEEYLVSHAIPCYSLDGDNVRHGLNRNLGFSPGDREENIRRIAEVAKLFADAGLVCITSFISPFAKDRENARKIHESAGLPFFEIFVDAPLNICESRDVKGLYKRARAGEIKGFTGIDSDYEKPETPERVLKTNLSTVSDCVHQVVELLQEQNIVPYTIIKDIHELFVPENKLDHVRAEAETLPSLSITKLDLQWVQVLSEGWATPLKGFMREKEYLQVMHFDTLLDDGVINMSIPIVLPVSAEDKTRLEGCSKFVLAHGGRRVAILRDAEFYEHRKEERCSRVWGTTCTKHPHIKMVMESGDWLVGGDLQVLEKIRWNDGLDQYRLTPLELKQKCKEMNADAVFAFQLRNPVHNGHALLMQDTRRRLLERGYKHPVLLLHPLGGWTKDDDVPLDWRMKQHAAVLEEGVLDPKSTIVAIFPSPMLYAGPTEVQWHCRSRMIAGANFYIVGRDPAGMPHPETKKDLYEPTHGGKVLSMAPGLTSVEIIPFRVAAYNKAKKAMDFYDPARHNEFDFISGTRMRKLAREGENPPDGFMAPKAWKVLTDYYRSLEKN

SEQ ID NO: 44 - protein, 382 aa, Homo sapiens; identical to SEQ ID NO: 5

SEQ ID NO: 45 - protein, 401 aa, Homo sapiens; identical to SEQ ID NO: 6

SEQ ID NO: 46 - protein, 147 aa, Homo sapiens; identical to SEQ ID NO: 7

SEQ ID NO: 47 - protein, 1211 aa, Homo sapiens; identical to SEQ ID NO: 8

SEQ ID NO: 48 - protein, 591 aa, Homo sapiens; identical to SEQ ID NO: 9

SEQ ID NO: 49 - protein, 98 aa, Homo sapiens; identical to SEQ ID NO: 10

SEQ ID NO: 50 - protein, 461 aa, Homo sapiens
MATFSRQEFFQQLLQGCLLPTAQQGLDQIWLLLAICLACRLLWRLGLPSYLKHASTVAGGFFSLYHFFQLHMVWVVLLSLLCYLVLFLCRHSSHRGVFLSVTILIYLLMGEMHMVDTVTWHKMRGAQMIVAMKAVSLGFDLDRGEVGTVPSPVEFMGYLYFVGTIVFGPWISFHSYLQAVQGRPLSCRWLQKVARSLALALLCLVLSTCVGPYLFPYFIPLNGDRLLRNKKRKARGTMVRWLRAYESAVSFHFSNYFVGFLSEATATLAGAGFTEEKDHLEWDLTVSKPLNVELPRSMVEVVTSWNLPMSYWLNNYVFKNALRLGTFSAVLVTYAASALLHGFSFHLAAVLLSLAFITYVEHVLRKRLARILSACVLSKRCPPDCSHQHRLGLGVRALNLLFGALAIFHLAYLGSLFDVDVDDTTEEQGYGMAYTVHKWSELSWASHWVTFGCWIFYRLIG

SEQ ID NO: 51 - protein, 541 aa, Homo sapiens
MPGLRRDRLLTLLLLGALLSADLYFHLWPQVQRQLRPRERPRGCPCTGRASSLARDSAAAASDPGTIVHNFSRTEPRTEPAGGSHSGSSSKLQALFAHPLYNVPEEPPLLGAEDSLLASQEALRYYRRKVARWNRRHKMYREQMNLTSLDPPLQLRLEASWVQFHLGINRHGLYSRSSPVVSKLLQDMRHFPTISADYSQDEKALLGACDCTQIVKPSGVHLKLVLRFSDFGKAMFKPMRQQRDEETPVDFFYFIDFQRHNAEIAAFHLDRILDFRRVPPTVGRIVNVTKEILEVTKNEILQSVFFVSPASNVCFFAKCPYMCKTEYAVCGNPHLLEGSLSAFLPSLNLAPRLSVPNPWIRSYTLAGKEEWEVNPLYCDTVKQIYPYNNSQRLLNVIDMAIFDFLIGNMDRHHYEMFTKFGDDGFLIHLDNARGFGRHSHDEISILSPLSQCCMIKKKTLLHLQLLAQADYRLSDVMRESLLEDQLSPVLTEPHLLALDRRLQTILRTVEGCIVAHGQQSVIVDGPVEQLAPDSGQANLTS

SEQ ID NO: 52 - protein, 376 aa, Homo sapiens
MCEEETTALVCDNGSGLCKAGFAGDDAPRAVFPSIVGRPRHQGVMVGMGQKDSYVGDEAQSKRGILTLKYPIEHGIITNWDDMEKIWHHSFYNELRVAPEEHPTLLTEAPLNPKANREKMTQIMFETFNVPAMYVAIQAVLSLYASGRTTGIVLDSGDGVTHNVPIYEGYALPHAIMRLDLAGRDLTDYLMKILTERGYSFVTTAEREIVRDIKEKLCYVALDFENEMATAASSSSLEKSYELPDGQVITIGNERFRCPETLFQPSFIGMESAGIHETTYNSIMKCDIDIRKDLYANNVLSGGTTMYPGIADRMQKEITALAPSTMKIKIIAPPERKYSVWIGGSILASLSTFQQMWISKPEYDEAGPSIVHRKCF

SEQ ID NO: 53 - protein, 744 aa, Homo sapiens; identical to SEQ ID NO: 14

SEQ ID NO: 54 - protein, 95 aa, Mus musculus; identical to SEQ ID NO: 15

SEQ ID NO: 55 - protein, 103 aa, Homo sapiens; identical to SEQ ID NO: 16

SEQ ID NO: 56 - protein, 471 aa, Homo sapiens; identical to SEQ ID NO: 17

SEQ ID NO: 57 - protein, 1170 aa, Homo sapiens; identical to SEQ ID NO: 18

SEQ ID NO: 58 - protein, 95 aa, Mus musculus; identical to SEQ ID NO: 19

SEQ ID NO: 59 - protein, 100 aa, Homo sapiens; identical to SEQ ID NO: 20

SEQ ID NO: 60 - protein, 95 aa, Mus musculus; identical to SEQ ID NO: 21

SEQ ID NO: 61 - protein, 377 aa, Homo sapiens; identical to SEQ ID NO: 22

SEQ ID NO: 62 - protein, 44 aa, Homo sapiens; identical to SEQ ID NO: 23

SEQ ID NO: 63 - protein, 455 aa, Homo sapiens
MRLLAWLIFLANWGGARAEPGKFWHIADLHLDPDYKVSKDPFQVCPSAGSQPVPDAGPWGDYLCDSPWALINSSIYAMKEIEPEPDFILWTGDDTPHVPDEKLGEAAVLEIVERLTKLIREVFPDTKVYAALGNHDFHPKNQFPAGSNNIYNQIAELWKPWLSNESIALFKKGAFYCEKLPGPSGAGRIVVLNTNLYYTSNALTADMADPGQQFQWLEDVLTDASKAGDMVYIVGHVPPGFFEKTQNKAWFREGFNEKYLKVVRKHHRVIAGQFFGHHHTDSFRMLYDDAGVPISAMFITPGVTPWKTTLPGVVNGANNPAIRVFEYDRATLSLKDMVTYFMNLSQANAQGTPRWELEYQLTEAYGVPDASAHSMHTVLDRIAGDQSTLQRYYVYNSVSYSAGVCDEACSMQHVCAMRQVDIDAYTTCLYASGTTPVPQLPLLLMALLGLCTLVL

SEQ ID NO: 64 - protein, 323 aa, Homo sapiens; identical to SEQ ID NO: 25

SEQ ID NO: 65 - protein, 483 aa, Homo sapiens
MTMPVNGAHKDADLWSSHDKMLAQPLKDSDVEVYNIIKKESNRQRVGLELIASENFASRAVLEALGSCLNNKYSEGYPGQRYYGGTEFIDELETLCQKRALQAYKLDPQCWGVNVQPYSGSPANFAVYTALVEPHGRIMGLDLPDGGHLTHGFMTDKKKISATSIFFESMPYKVNPDTGYINYDQLEENARLFHPKLIIAGTSCYSRNLEYARLRKIADENGAYLMADMAHISGLVAAGVVPSPFEHCHVVTTTTHKTLRGCRAGMIFYRKGVKSVDPKTGKEILYNLESLINSAVFPGLQGGPHNHAIAGVAVALKQAMTLEFKVYQHQVVANCRALSEALTELGYKIVTGGSDNHLILVDLRSKGTDGGRAEKVLEACSIACNKNTCPGDRSALRPSGLRLGTPALTSRGLLEKDFQKVAHFIHRGIELTLQIQSDTGVRATLKEFKERLAGDKYQAAVQALREEVESFASLFPLPGLPDF

SEQ ID NO: 66 - protein, 475 aa, Homo sapiens; identical to SEQ ID NO: 27

SEQ ID NO: 67 - protein, 1887 aa, Homo sapiens; identical to SEQ ID NO: 28

SEQ ID NO: 68 - protein, 330 aa, Homo sapiens
MPHSVTLRGPSPWGFRLVGGRDFSAPLTISRVHAGSKAALAALCPGDLIQAINGESTELMTHLEAQNRIKGCHDHLTLSVSRPEGRSWPSAPDDSKAQAHRIHIDPEIQDGSPTTSRRPSGTGTGPEDGRPSLGSPYGQPPRFPVPHNGSSEATLPAQMSTLHVSPPPSADPARGLPRSRDCRVDLGSEVYRMLREPAEPVAAEPKQSGSFRYLQGMLEAGEGGDWPGPGGPRNLKPTASKLGAPLSGLQGLPECTRCGHGIVGTIVKARDKLYHPECFMCSDCGLNLKQRGYFFLDERLYCESHAKARVKPPEGYDVVAVYPNAKVELV

SEQ ID NO: 69 - protein, 262 aa, Homo sapiens; identical to SEQ ID NO: 30

SEQ ID NO: 70 - protein, 700 aa, Homo sapiens
MSRRTRCEDLDELHYQDTDSDVPEQRDSKCKVKWTHEEDEQLRALVRQFGQQDWKFLASHFPNRTDQQCQYRWLRVLNPDLVKGPWTKEEDQKVIELVKKYGTKQWTLIAKHLKGRLGKQCRERWHNHLNPEVKKSCWTEEEDRIICEAHKVLGNRWAEIAKMLPGRTDNAVKNHWNSTIKRKVDTGGFLSESKDCKPPVYLLLELEDKDGLQSAQPTEGQGSLLTNWPSVPPTIKEEENSEEELAAATTSKEQEPIGTDLDAVRTPEPLEEFPKREDQEGSPPETSLPYKWVVEAANLLIPAVGSSLSEALDLIESDPDAWCDLSKFDLPEEPSAEDSINNSLVQLQASHQQQVLPPRQPSALVPSVTEYRLDGHTISDLSRSSRGELIPISPSTEVGGSGIGTPPSVLKRQRKRRVALSPVTENSTSLSFLDSCNSLTPKSTPVKTLPFSPSQFLNFWNKQDTLELESPSLTSTPVCSQKVVVTTPLHRDKTPLHQKHAAFVTPDQKYSMDNTPHTPTPFKNALEKYGPLKPLPQTPHLEEDLKEVLRSEAGIELIIEDDIRPEKQKRKPGLRRSPIKKVRKSLALDIVDEDVKLMMSTLPKSLSLPTTAPSNSSSLTLSGIKEDNSLLNQGFLQAKPEKAAVAQKPRSHFTTPAPMSSAWKTVACGGTRDQLFMQEKARQLLGRLKPSHTSRTLILS

SEQ ID NO: 71 - protein, 210 aa, Homo sapiens
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQFYK

SEQ ID NO: 72 - protein, 177 aa, Mus musculus; identical to SEQ ID NO: 33

SEQ ID NO: 73 - protein, 558 aa, Homo sapiens
MEAAAQFFVESPDVVYGPEAIEAQYEYRTTRVSREGGVLKVHPTSTRFTFRTARQVPRLGVMLVGWGGNNGSTLTAAVLANRLRLSWPTRSGRKEANYYGSLTQAGTVSLGLDAEGQEVFVPFSAVLPMVAPNDLVFDGWDISSLNLAEAMRRAKVLDWGLQEQLWPHMEALRPRPSVYIPEFIAANQSARADNLIPGSRAQQLEQIRRDIRDFRSSAGLDKVIVLWTANTERFCEVIPGLNDTAENLLRTIELGLEVSPSTLFAVASILEGCAFLNGSPQNTLVPGALELAWQHRVFVGGDDFKSGQTKVKSVLVDFLIGSGLKTMSIVSYNHLGNNDGENLSAPLQFRSKEVSKSNVVDDMVQSNPVLYTPGEEPDHCVVIKYVPYVGDSKRALDEYTSELMLGGTNTLVLHNTCEDSLLAAPIMLDLALLTELCQRVSFCTDMDPEPQTFHPVLSLLSFLFKAPLVPPGSPVVNALFRQRSCIENILRACVGLPPQNHMLLEHKMERPGPSLKRVGPVAATYPMLNKKGPVPAATNGCTGDANGHLQEEPPMPTT

SEQ ID NO: 74 - protein, 113 aa, Homo sapiens
MAGIRALFMYLWLQLDWVSRGESVGLHLPTLSVQEGDNSIINCAYSNSASDYFIWYKQESGKGPQFIIDIRSNMDKRQGQRVTVLLNKTVKHLSLQIAATQPGDSAVYFCAEN

SEQ ID NO: 75 - protein, 354 aa, Homo sapiens; identical to SEQ ID NO: 35

SEQ ID NO: 76 - protein, 116 aa, Mus musculus
MWQFCILCLCVLMASVATDPTVTLLEQNPRWRLVPRGQAVNLRCILKNSQYPWMSWYQQDLQKQLQWLFTLRSPGDKEVKSLPGADYLATRVTDTELRLQVANMSQGRTLYCTCSA

SEQ ID NO: 77 - protein, 161 aa, Homo sapiens; identical to SEQ ID NO: 36

SEQ ID NO: 78 - protein, 276 aa, Mus musculus; identical to SEQ ID NO: 37

SEQ ID NO: 79 - protein, 766 aa, Homo sapiens; identical to SEQ ID NO: 38

SEQ ID NO: 80 - protein, 396 aa, Homo sapiens
MKTLLLLLLVLLELGEAQGSLHRVPLRRHPSLKKKLRARSQLSEFWKSHNLDMIQFTESCSMDQSAKEPLINYLDMEYFGTISIGSPPQNFTVIFDTGSSNLWVPSVYCTSPACKTHSRFQPSQSSTYSQPGQSFSIQYGTGSLSGIIGADQVSVEGLTVVGQQFGESVTEPGQTFVDAEFDGILGLGYPSLAVGGVTPVFDNMMAQNLVDLPMFSVYMSSNPEGGAGSELIFGGYDHSHFSGSLNWVPVTKQAYWQIALDNIQVGGTVMFCSEGCQAIVDTGTSLITGPSDKIKQLQNAIGAAPVDGEYAVECANLNVMPDVTFTINGVPYTLSPTAYTLLDFVDGMQFCSSGFQGLDIHPPAGPLWILGDVFIRQFYSVFDRGNNRVGLAPAVP

SEQ ID NO: 81 - protein, 182 aa, Homo sapiens; identical to SEQ ID NO: 40

SEQ ID NO: 82 - protein, 454 aa, Mus musculus
SQSFPNVFPLVSCESPLSDKNLVAMGCLARDFLPSTISFTWNYQNNTEVIQGIRTFPTLRTGGKYLATSQVLLSPKSILEGSDEYLVCKIHYGGKNRDLHVPIPAVAEMNPNVNVFVPPRDGFSGPAPRKSKLICEATNFTPKPITVSWLKDGKLVESGFTTDPVTIENKGSTPQTYKVISTLTISEIDWLNLNVYTCRVDHRGLTFLKNVSSTCAASPSTDILTFTIPPSFADIFLSKSANLTCLVSNLATYETLNISWASQSGEPLETKIKIMESHPNGTFSAKGVASVCVEDWNNRKEFVCTVTHRDLPSPQKKFISKPNEVHKHPPAVYLLPPAREQLNLRESATVTCLVKGFSPADISVQWLQRGQLLPQEKYVTSAPMPEPGAPGFYFTHSILTVTEEEWNSGETYTCVVGHEALPHLVTERTVDKSTGKPTLYNVSLIMSDTGGTCY

SEQ ID NO: 83 - protein, 140 aa, Mus musculus
MIGLVTKFQIYCSHSPWYGTSHTSIYFCYPGMLGQSATHGSLSSRTLFFPSSRGQKCGPQQALLCPQPTILGWCCFKLVIWVLSQSSEAIAVGQSLSPGTNIPVTVCPLYLMPCFKNPVLLGLMPTLIQHKFISTRFHPP

SEQ ID NO: 84 - protein, 318 aa, Homo sapiens
MPKRGKKGAVAEDGDELRTEPEAKKSKTAAKKNDKEAAGEGPALYEDPPDQKTSPSGKPATLKICSWNVDGLRAWIKKKGLDWVKEEAPDILCLQETKCSENKLPAELQELPGLSHQYWSAPSDKEGYSGVGLLSRQCPLKVSYGIGDEEHDQEGRVIVAEFDSFVLVTAYVPNAGRGLVRLEYRQRWDEAFRKFLKGLASRKPLVLCGDLNVAHEEIDLRNPKGNKKNAGFTPQERQGFGELLQAVPLADSFRHLYPNTPYAYTFWTYMMNARSKNVGWRLDYFLLSHSLLPALCDSKIRSKALGSDHCPITLYLAL

SEQ ID NO: 85 - DNA, 1558 bp, Homo sapiens
cagacagaccaatcacgcgcattcttcggccacgacaagcgcgcctctgatcacgtgaccaggtccgctacccacgtgggggctcagcgtgcacccttctttgtgctcgggttaggaggagctaggctgccatcgggccggtgcagatacggggttgctcttttgctcataagaggggcttcgctggcagtctgaacggcaagcttgagtcaggacccttaattaagatcctcaattggctggagggcagatctcgcgagcaacgcggtaaaaatattgcttcggtgggtgacgcggtacagctgcccaagggcgttcgtaacgggaatgccgaagcgtgggaaaaagggagcggtggcggaagacggggatgagctcaggacagagccagaggccaagaagagtaagacggccgcaaagaaaaatgacaaagaggcagcaggagagggcccagccctgtatgaggaccccccagatcagaaaacctcacccagtggcaaacctgccacactcaagatctgctcttggaatgtggatgggcttcgagcctggattaagaagaaaggattagattgggtaaaggaagaagccccagatatactgtgccttcaagagaccaaatgttcagagaacaaactaccagctgaacttcaggagctgcctggactctctcatcaatactggtcagctccttcggacaaggaagggtacagtggcgtgggcctgctttcccgccagtgcccactcaaagtttcttacggcataggcgatgaggagcatgatcaggaaggccgggtgattgtggctgaatttgactcgtttgtgctggtaacagcatatgtacctaatgcaggccgaggtctggtacgactggagtaccggcagcgctgggatgaagcctttcgcaagttcctgaagggcctggcttcccgaaagccccttgtgctgtgtggagacctcaatgtggcacatgaagaaattgaccttcgcaaccccaaggggaacaaaaagaatgctggcttcacgccacaagagcgccaaggcttcggggaattactgcaggctgtgccactggctgacagctttaggcacctctaccccaacacaccctatgcctacaccttttggacttatatgatgaatgctcgatccaagaatgttggttggcgccttgattactttttgttgtcccactctctgttacctgcattgtgtgacagcaagatccgttccaaggccctcggcagtgatcactgtcctatcaccctatacctagcactgtgacaccacccctaaatcactttgagcctgggaaataagccccctcaactaccattccttctttaaacactcttcagagaaatctgcattctatttctcatgtataaaactaggaatcctccaaccaggctcctgtgatagagttcttttaagcccaagattttttatttgagggttttttgttttttaaaaaaaaattgaacaaagactactaatgactttgtttgaattatccacatgaaaataaagagccatagtttcagccttaaaaaaaaaa

SEQ ID NO: 86 - protein, 318 aa, Homo sapiens; identical to SEQ ID NO: 84

SEQ ID NO: 87 - DNA, 1563 bp, Homo sapiens
cagacagaccaatcacgcgcattcttcggccacgacaagcgcgcctctgatcacgtgaccaggtccgctacccacgtgggggctcagcgtgcacccttctttgtgctcgggttaggaggagctaggctgccatcgggccggtgcagatacggggttgctcttttgctcataagaggggcttcgctggcagtctgaacggcaagcttgagtcaggacccttaattaagatcctcaattggctggagggcagatctcgcgagtagggcaacgcggtaaaaatattgcttcggtgggtgacgcggtacagctgcccaagggcgttcgtaacgggaatgccgaagcgtgggaaaaagggagcggtggcggaagacggggatgagctcaggacagagccagaggccaagaagagtaagacggccgcaaagaaaaatgacaaagaggcagcaggagagggcccagccctgtatgaggaccccccagatcagaaaacctcacccagtggcaaacctgccacactcaagatctgctcttggaatgtggatgggcttcgagcctggattaagaagaaaggattagattgggtaaaggaagaagccccagatatactgtgccttcaagagaccaaatgttcagagaacaaactaccagctgaacttcaggagctgcctggactctctcatcaatactggtcagctccttcggacaaggaagggtacagtggcgtgggcctgctttcccgccagtgcccactcaaagtttcttacggcataggcgatgaggagcatgatcaggaaggccgggtgattgtggctgaatttgactcgtttgtgctggtaacagcatatgtacctaatgcaggccgaggtctggtacgactggagtaccggcagcgctgggatgaagcctttcgcaagttcctgaagggcctggcttcccgaaagccccttgtgctgtgtggagacctcaatgtggcacatgaagaaattgaccttcgcaaccccaaggggaacaaaaagaatgctggcttcacgccacaagagcgccaaggcttcggggaattactgcaggctgtgccactggctgacagctttaggcacctctaccccaacacaccctatgcctacaccttttggacttatatgatgaatgctcgatccaagaatgttggttggcgccttgattactttttgttgtcccactctctgttacctgcattgtgtgacagcaagatccgttccaaggccctcggcagtgatcactgtcctatcaccctatacctagcactgtgacaccacccctaaatcactttgagcctgggaaataagccccctcaactaccattccttctttaaacactcttcagagaaatctgcattctatttctcatgtataaaactaggaatcctccaaccaggctcctgtgatagagttcttttaagcccaagattttttatttgagggttttttgttttttaaaaaaaaattgaacaaagactactaatgactttgtttgaattatccacatgaaaataaagagccatagtttcagccttaaaaaaaaaa

SEQ ID NO: 88 - protein, 318 aa, Homo sapiens; identical to SEQ ID NO: 84

SEQ ID NO: 89 - DNA, 1497 bp, Homo sapiens
cagacagaccaatcacgcgcattcttcggccacgacaagcgcgcctctgatcacgtgaccaggtccgctacccacgtgggggctcagcgtgcacccttctttgtgctcgggttaggaggagctaggctgccatcgggccggtgcagatacggggttgctcttttgctcataagaggggcttcgctggcagtctgaacggcaacgcggtaaaaatattgcttcggtgggtgacgcggtacagctgcccaagggcgttcgtaacgggaatgccgaagcgtgggaaaaagggagcggtggcggaagacggggatgagctcaggacagagccagaggccaagaagagtaagacggccgcaaagaaaaatgacaaagaggcagcaggagagggcccagccctgtatgaggaccccccagatcagaaaacctcacccagtggcaaacctgccacactcaagatctgctcttggaatgtggatgggcttcgagcctggattaagaagaaaggattagattgggtaaaggaagaagccccagatatactgtgccttcaagagaccaaatgttcagagaacaaactaccagctgaacttcaggagctgcctggactctctcatcaatactggtcagctccttcggacaaggaagggtacagtggcgtgggcctgctttcccgccagtgcccactcaaagtttcttacggcataggcgatgaggagcatgatcaggaaggccgggtgattgtggctgaatttgactcgtttgtgctggtaacagcatatgtacctaatgcaggccgaggtctggtacgactggagtaccggcagcgctgggatgaagcctttcgcaagttcctgaagggcctggcttcccgaaagccccttgtgctgtgtggagacctcaatgtggcacatgaagaaattgaccttcgcaaccccaaggggaacaaaaagaatgctggcttcacgccacaagagcgccaaggcttcggggaattactgcaggctgtgccactggctgacagctttaggcacctctaccccaacacaccctatgcctacaccttttggacttatatgatgaatgctcgatccaagaatgttggttggcgccttgattactttttgttgtcccactctctgttacctgcattgtgtgacagcaagatccgttccaaggccctcggcagtgatcactgtcctatcaccctatacctagcactgtgacaccacccctaaatcactttgagcctgggaaataagccccctcaactaccattccttctttaaacactcttcagagaaatctgcattctatttctcatgtataaaactaggaatcctccaaccaggctcctgtgatagagttcttttaagcccaagattttttatttgagggttttttgttttttaaaaaaaaattgaacaaagactactaatgactttgtttgaattatccacatgaaaataaagagccatagtttcagccttaaaaaaaaaa

SEQ ID NO: 90 - protein, 318 aa, Homo sapiens; identical to SEQ ID NO: 84

SEQ ID NO: 91 - DNA, 1507 bp, Homo sapiens
cagacagaccaatcacgcgcattcttcggccacgacaagcgcgcctctgatcacgtgaccaggtccgctacccacgtgggggctcagcgtgcacccttctttgtgctcgggttaggaggagctaggctgccatcgggccggtgcagatacggggttgctcttttgctcataagaggggcttcgctggcagtctgaacggcaagcttgagcaacgcggtaaaaatattgcttcggtgggtgacgcggtacagctgcccaagggcgttcgtaacgggaatgccgaagcgtgggaaaaagggagcggtggcggaagacggggatgagctcaggacagagccagaggccaagaagagtaagacggccgcaaagaaaaatgacaaagaggcagcaggagagggcccagccctgtatgaggaccccccagatcagaaaacctcacccagtggcaaacctgccacactcaagatctgctcttggaatgtggatgggcttcgagcctggattaagaagaaaggattagattgggtaaaggaagaagccccagatatactgtgccttcaagagaccaaatgttcagagaacaaactaccagctgaacttcaggagctgcctggactctctcatcaatactggtcagctccttcggacaaggaagggtacagtggcgtgggcctgctttcccgccagtgcccactcaaagtttcttacggcataggcgatgaggagcatgatcaggaaggccgggtgattgtggctgaatttgactcgtttgtgctggtaacagcatatgtacctaatgcaggccgaggtctggtacgactggagtaccggcagcgctgggatgaagcctttcgcaagttcctgaagggcctggcttcccgaaagccccttgtgctgtgtggagacctcaatgtggcacatgaagaaattgaccttcgcaaccccaaggggaacaaaaagaatgctggcttcacgccacaagagcgccaaggcttcggggaattactgcaggctgtgccactggctgacagctttaggcacctctaccccaacacaccctatgcctacaccttttggacttatatgatgaatgctcgatccaagaatgttggttggcgccttgattactttttgttgtcccactctctgttacctgcattgtgtgacagcaagatccgttccaaggccctcggcagtgatcactgtcctatcaccctatacctagcactgtgacaccacccctaaatcactttgagcctgggaaataagccccctcaactaccattccttctttaaacactcttcagagaaatctgcattctatttctcatgtataaaactaggaatcctccaaccaggctcctgtgatagagttcttttaagcccaagattttttatttgagggttttttgttttttaaaaaaaaattgaacaaagactactaatgactttgtttgaattatccacatgaaaataaagagccatagtttcagccttaaaaaaaaaa

SEQ ID NO: 92 - protein, 317 aa, Mus musculus
MPKRGKKAAADDGEEPKSEPETKKSKGAAKKTEKEAAGEGPVLYEDPPDQKTSPSGKSATLKICSWNVDGLRAWIKKKGLDWVKEEAPDILCLQETKCSENKLPAELQELPGLTHQYWSAPSDKEGYSGVGLLSRQCPLKVSYGIGEEEHDQEGRVIVAEFESFVLVTAYVPNAGRGLVRLEYRQRWDEAFRKFLKDLASRKPLVLCGDLNVAHEEIDLRNPKGNKKNAGFTPQERQGFGELLQAVPLADSFRHLYPNTAYAYTFWTYMMNARSKNVGWRLDYFLLSHSLLPALCDSKIRSKALGSDHCPITLYLAL

SEQ ID NO: 93 - DNA, 1307 bp, Mus musculus
cacgtgaccacaccaatcgcccacgtgggaacaggcagactccattctttgtgccgtgagggtctctggcttcgttgggaggcagcgcagtaaacactgcttcggtgctccaggagcctaagggctttcgtcacagcgatgccaaagcggggaaagaaagcggcggccgacgacggggaagaacccaagtcggagccagagaccaagaagagtaagggggcagcaaagaaaaccgagaaggaggccgcgggagagggccctgtcctgtacgaggaccctccagatcagaaaacctcacccagtggcaaatctgccacactcaagatatgctcctggaatgtggatgggcttcgagcctggattaaaaagaaaggtttggattgggtaaaggaagaagcaccagatatcttgtgcctccaagagaccaagtgctcggagaacaaactcccggctgaactgcaagagctgcctggactcacccatcagtactggtcagctccgtcagacaaagaaggatacagtggtgtgggcctactttcccgccagtgcccgctaaaagtctcttatggcattggcgaggaagaacatgatcaagaaggccgggtgattgtggctgaatttgagtcctttgtcctggtaacagcctatgttcccaatgcaggcaggggtctggtaagactggaataccgacagcgttgggatgaagccttccgaaagtttctaaaggacttggcttccagaaagcctcttgtgctatgtggggatctcaatgtggctcatgaagaaattgacctccgtaaccccaaaggaaacaaaaagaatgctggctttactccccaggagcgccaaggttttggggaactgctacaagctgtaccattggctgacagcttccggcatctctaccccaacactgcttacgcttacactttctggacttacatgatgaatgcccgctctaagaatgttggttggcgccttgattactttttgctttcccactctcttttacctgcattgtgtgacagcaagatccggtccaaggctcttggcagtgaccactgtcccatcaccctttacctagcactgtgataccctcctgcagtagcttcctgcctgggagatggctctctctgcagaagtctggtgttttagccttcaggtgtttggttttgtatgtgctccctcattttaaacattaaaccaaacttctggtttcctttagacaatccaagagaaataaaaggccctactttcagcttggctttctttgtccctttgtgaaaactacatattccctttttcat

(19) United States
(12) Patent Application Publication
(10) Pub. No.: US 2018/0305776 A1
(43) Pub. Date: Oct. 25, 2018

(54) MYC-REPORTERS, CELLS CONTAINING MYC-REPORTERS AND METHODS OF USE THEREOF

(71) Applicant: The Board of Trustees of The Leland Stanford Junior University, Stanford, CA (US)

(72) Inventors: Anja Deutzmann, Stanford, CA (US); Dean W. Felsher, San Mateo, CA (US); Yulin Li, Stanford, CA (US)

(21) Appl. No.: 15/957,816

(22) Filed: Apr. 19, 2018

Related U.S. Application Data
(60) Provisional application No. 62/487,955, filed on Apr. 20, 2017.

(51) Int. Cl. C12Q 1/6897; G01N 33/50; A01K 67/027

(52) U.S. Cl. CPC C12Q 1/6897; G01N 33/5011; A01K 2217/203; C12Q 2600/136; A01K 2267/0331; A01K 67/0275

(57) ABSTRACT

Provided are MYC-reporters and MYC-reporter expression vectors having MYC-reporter activity. Also provided are cells containing MYC-reporters and/or MYC-reporter expression vectors as well as animals containing such cells and/or genetically modified to contain one or more MYC-reporters and/or expression vectors. Also provided are methods of screening, including methods of screening a candidate agent for MYC repression. The subject methods may, in some instances, employ one or more of the provided MYC-reporters, expression vectors, cells and/or transgenic animals.

### CROSS-REFERENCE TO RELATED APPLICATIONS

Pursuant to 35 U.S.C. § 119(e), this application claims priority to the filing date of the U.S. Provisional Patent Application Ser. No. 62/487,955, filed Apr. 20, 2017, the disclosure of which application is herein incorporated by reference.

### GOVERNMENT RIGHTS

This invention was made with Government support under contracts CA089305, CA114747, CA143907, CA149145, CA170378, CA184384, and CA188383 awarded by the National Institutes of Health. The Government has certain rights in the invention.

### INCORPORATION BY REFERENCE OF SEQUENCE LISTING PROVIDED AS A TEXT FILE

A Sequence Listing is provided herewith as a text file, “STAN-1360_SeqList_ST25.txt” created on Apr. 19, 2018 and having a size of 306 KB. The contents of the text file are incorporated by reference herein in their entirety.

### BACKGROUND

The MYC oncogene is a transcription factor that regulates expression of thousands of gene products. These in turn regulate many cellular programs including proliferation, growth, self-renewal, differentiation, angiogenesis, and host immune response. The MYC oncogene is overexpressed and causally associated with most types of human cancer including both hematopoietic tumors such as Burkitt's lymphoma, diffuse large cell lymphoma and T-cell acute lymphocytic leukemia (T-ALL), and solid tumors such as hepatocellular carcinoma (HCC), osteosarcoma (OS), lung adenocarcinoma (LC), and renal cell carcinoma (RCC).

Burkitt lymphoma affects both children and adults. While it makes up over a third of all the lymphomas seen in children, the number of people over 60 who are diagnosed with this type of lymphoma is also increasing. However, solid tumors represent the majority of cancer types. In 2013, in the United States, 9 of top 10 invasive cancers in terms of incidence were solid tumors. HCC, in particular, is a common highly fatal cancer, of which incidences are increasing. There is no effective therapy for HCC patients whose disease is not surgically resectable. Existing targeting therapeutics have had only marginal efficacy.

### SUMMARY

Provided are MYC-reporters and MYC-reporter expression vectors having MYC-reporter activity. Also provided are cells containing MYC-reporters and/or MYC-reporter expression vectors as well as animals containing such cells and/or genetically modified to contain one or more MYC-reporters and/or expression vectors. Also provided are methods of screening, including methods of screening a candidate agent for MYC repression. The subject methods may, in some instances, employ one or more of the provided MYC-reporters, expression vectors, cells and/or transgenic animals.

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1A-1B. Transgenic mouse model of MYC-induced cancer. A) The tetracycline transactivator (tTA) is expressed under a tissue-specific promoter. MYC is expressed under the control of the tetracycline-responsive element (TRE). MYC expression in the target tissue can be regulated by administration of doxycycline (DOX). In the absence of DOX, MYC is expressed (MYC ON) and mice develop tissue-specific tumors. In the presence of DOX, MYC expression is inactivated (MYC OFF) and tumors regress. B) Cell lines can be derived from MYC-induced tumors and MYC expression can be manipulated in in vitro cultures. In a cell line derived from MYC-induced osteosarcoma MYC inactivation leads to loss of proliferative potential and differentiation.

FIG. 2A-2C. In vitro and in vivo efficacy evaluation of a novel MYC-inhibitor candidate agent. A) Immunofluorescence images of a MYC-driven osteosarcoma cell line treated with a candidate MYC inhibitor (MYCi candidate) or with doxycycline to inactivate MYC expression. Specific antibodies were used to visualize the cytoskeleton and the expression of a MYC target genes to assess cell morphology and MYC activity. B) NSG mice bearing MYC-driven luciferase-labeled T-ALL were treated with vehicle or the MYCi candidate. Tumor burden was assessed by bioluminescence imaging (BLI) after 12 days of treatment and is represented as average radiance. C) NSG mice with sub-cutaneous transplants of MYC-driven luciferase-labeled HCC were treated (IP) with vehicle or MYCi candidate. Tumor burden was quantified by BLI after 21 days and is shown as average radiance.

FIG. 3A-3B. Generation of a reporter cell line for the identification of MYC inhibitors. A) Expression changes upon MYC shut off in MYC-driven osteo-sarcoma cell line. Forty proteins with highest expression changes (proteins driven by Class I promoters: 20 up; proteins driven by Class 2 promoters including Ebox tandem promoters: 20 down) are shown. Red: high, Blue: low expression. B) A tet-regulated MYC-driven osteosarcoma cell line is genetically engineered to express fluorescently-labeled cytoskeleton to visualize morphological changes associated with differentiation upon MYC inactivation. The cells further express fluorescent transcription reporters: Fluorescent protein 1 (FP1) is transcribed in the presence and fluorescent protein 2 (FP2) in the absence of MYC expression. Both reporters may be targeted to the nucleus by a nuclear leading sequence (NLS).

### DEFINITIONS

The term “gene” refers to a particular unit of heredity present at a particular locus within the genetic component of an organism. A gene may be a nucleic acid sequence, e.g., a DNA or RNA sequence, present in a nucleic acid genome, a DNA or RNA genome, of an organism and, in some instances, may be present on a chromosome. Typically a gene will be a DNA sequence that encodes for an mRNA that encodes a protein. A gene may be comprised of a single exon and no introns or multiple exons and one or more introns. One of two or more identical or alternative forms of a gene present at a particular locus is referred to as an “allele” and, for example, a diploid organism will typically have two alleles of a particular gene. New alleles of a particular gene may be generated either naturally or artificially through natural or induced mutation and propagated through breeding or cloning. A gene or allele may be isolated from the genome of an organism and replicated and/or manipulated or a gene or allele may be modified in situ through gene therapy methods. The locus of a gene or allele may have associated regulatory elements and gene therapy, in some instances, may include modification of the regulatory elements of a gene or allele while leaving the coding sequences of the gene or allele unmodified.

“Operably linked” refers to a juxtaposition wherein the components so described are in a relationship permitting them to function in their intended manner. For instance, a promoter is operably linked to a coding sequence if the promoter affects its transcription or expression.

The terms “specific binding,” “specifically binds,” and the like, refer to non-covalent or covalent preferential binding to a molecule relative to other molecules or moieties in a solution or reaction mixture (e.g., an antibody specifically binds to a particular polypeptide or epitope relative to other available polypeptides). In some embodiments, the affinity of one molecule for another molecule to which it specifically binds is characterized by a K_{D} (dissociation constant) of 10⁻⁵ M or less (e.g., 10⁻⁶ M or less, 10⁻⁷ M or less, 10⁻⁸ M or less, 10⁻⁹ M or less, 10⁻¹⁰ M or less, 10⁻¹¹ M or less, 10⁻¹² M or less, 10⁻¹³ M or less, 10⁻¹⁴ M or less, 10⁻¹⁵ M or less, or 10⁻¹⁶ M or less). “Affinity” refers to the strength of binding, increased binding affinity being correlated with a lower K_{D}.

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom(s) but has not yet been diagnosed as having it; (b) inhibiting the disease and/or symptom(s), i.e., arresting development of a disease and/or the associated symptoms; or (c) relieving the disease and the associated symptom(s), i.e., causing regression of the disease and/or symptom(s). Those in need of treatment can include those already inflicted (e.g., those with cancer, e.g. those having tumors) as well as those in which prevention is desired (e.g., those with increased susceptibility to cancer; those with cancer; those suspected of having cancer; etc.).

The terms “recipient”, “individual”, “subject”, “host”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, camels, etc. In some embodiments, the mammal is a human, a mouse or a rat.

The terms “co-administration” and “in combination with” include the administration of two or more therapeutic agents either simultaneously, concurrently or sequentially within no specific time limits. In one embodiment, the agents are present in the cell or in the subjects body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

A “therapeutically effective amount”, a “therapeutically effective dose” or “therapeutic dose” is an amount sufficient to effect desired clinical results (i.e., achieve therapeutic efficacy, achieve a desired therapeutic response, etc.). A therapeutically effective dose can be administered in one or more administrations. For purposes of this disclosure, a therapeutically effective dose of an agent that inhibits a target gene (e.g., a MYC-dependent target gene, and the like) and/or compositions is an amount that is sufficient, when administered to the individual, to palliate, ameliorate, stabilize, reverse, prevent, slow or delay the progression of the disease state (e.g., cancer, etc.) by, for example, inhibiting the growth of, inducing death of or otherwise preventing the clinical progressing of a cancer with elevated MYC levels/expression present in the subject.

The terms “control”, “control reaction”, “control assay”, and the like, refer to a reaction, test, or other portion of an experimental or diagnostic procedure or experimental design for which an expected result is known with high certainty, e.g., in order to indicate whether the results obtained from associated experimental samples are reliable, indicate to what degree of confidence associated experimental results indicate a true result, and/or to allow for the calibration of experimental results. For example, in some instances, a control may be a “negative control” such that an essential component of the assay is excluded from the negative control reaction such that an experimenter may have high certainty that the negative control reaction will not produce a positive result. In some instances, a control may be “positive control” such that all components of a particular assay are characterized and known, when combined, to produce a particular result in the assay being performed such that an experimenter may have high certainty that the positive control reaction will not produce a negative result.

### DETAILED DESCRIPTION

Provided are MYC-reporters and MYC-reporter expression vectors having MYC-reporter activity. Also provided are cells containing MYC-reporters and/or MYC-reporter expression vectors as well as animals containing such cells and/or genetically modified to contain one or more MYC-reporters and/or expression vectors. Also provided are methods of screening, including methods of screening a candidate agent for MYC repression. The subject methods may, in some instances, employ one or more of the provided MYC-reporters, expression vectors, cells and/or transgenic animals.

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Reporters and Cells

As summarized above, the present disclosure provides MYC-reporters and MYC-reporter expression vectors having MYC-reporter activity, as well as cells containing such MYC-reporters and/or MYC-reporter expression vectors. The present disclosure also provides animals containing such cells and/or genetically modified to contain one or more MYC-reporters and/or expression vectors.

By “MYC-reporter” is generally meant a nucleic acid construct that provides a readout of MYC activity, e.g., within a cell in vitro or in vivo. The subject MYC-reporters will generally include a sequence encoding a detectable reporter that may be visually, optically or otherwise detected as a readout of MYC activity in a cell. In some instances, a signal from a detectable reporter may be quantified.

The sequence encoding the detectable reporter will generally be operably linked to a regulatory sequence of a MYC-responsive gene or other MYC-responsive element. Useful regulatory sequences include but are not limited to e.g., promoters, enhancers, etc. Useful MYC-responsive elements include but are not limited to e.g., Ebox containing elements, such as e.g., tandem Ebox elements. In some instances, useful regulatory sequences may include a regulatory sequence of one or more of the following MYC-responsive genes: expressed sequence AI593442 (AI593442); 3′-phosphoadenosine 5′-phosphosulfate synthase 2 (PAPSS2); proline and arginine rich end leucine rich repeat protein (PRELP); TNF receptor superfamily member 11 b (TNFRSF11B); ribonuclease A family member 4 (RNASE4); ADAM metallopeptidase with thrombospondin type 1 motif 2 (ADAMTS2); guanylate binding protein 2 (GBP2); S100 calcium binding protein A13 (S100A13); porcupine homolog (PORCN); FAM20A, golgi associated secretory pathway pseudokinase (FAM20A); actin, gamma 2, smooth muscle, enteric (ACTG2); collagen type VIII alpha 1 chain (COL8A1); bone gamma-carboxyglutamate protein 3 (BGLAP-RS1); matrix Gla protein (MGP); matrix metallopeptidase 13 (MMP13); thrombospondin 1 (THBS1); bone gamma carboxyglutamate protein (BGLAP1); bone gamma-carboxyglutamate protein 2 (BGLAP2); actin, alpha 2, smooth muscle, aorta (ACTA2); thymosin beta 4, X-linked (TMSB4X); sphingomyelin phosphodiesterase acid like 3B (SMPDL3B); YdjC homolog (YDJC); serine hydroxymethyltransferase 1 (SHMT1); lipoprotein lipase (LPL); nucleoporin 210 (NUP210); PDZ and LIM domain 4 (PDLIM4); lysosomal protein transmembrane 5 (LAPTM5); MYB proto-oncogene like 2 (MYBL2); CD8b molecule (CD8B1); erythroid differentiation regulator 1 (ERDR1); inositol-3-phosphate synthase 1 (ISYNA1); T cell receptor beta, variable 13-2 (TCRB-V8.2); carbonic anhydrase 12 (CAR12); T cell receptor beta variable 1 (TRBV1); Thy-1 cell surface antigen (THY1); cyclin B1 interacting protein 1 (GM288); dipeptidyl peptidase 4 (DPP4); cathepsin E (CTSE); CD3g molecule (CD3G) and immunoglobulin heavy constant mu (IGH-6).

In some instances, a useful regulatory sequence may be a regulatory sequence that is downregulated by MYC. In some instances, regulatory sequences downregulated by MYC may include what are referred to herein as “Class I promoters” or the regulatory sequence of what is referred to herein as a “Class I gene”. Useful regulatory sequences that are downregulated by MYC include but are not limited to e.g., a regulatory sequence of one or more of the following MYC-downregulated genes: expressed sequence AI593442 (AI593442); 3′-phosphoadenosine 5′-phosphosulfate synthase 2 (PAPSS2); proline and arginine rich end leucine rich repeat protein (PRELP); TNF receptor superfamily member 11b (TNFRSF11B); ribonuclease A family member 4 (RNASE4); ADAM metallopeptidase with thrombospondin type 1 motif 2 (ADAMTS2); guanylate binding protein 2 (GBP2); S100 calcium binding protein A13 (S100A13); porcupine homolog (PORCN); FAM20A, golgi associated secretory pathway pseudokinase (FAM20A); actin, gamma 2, smooth muscle, enteric (ACTG2); collagen type VIII alpha 1 chain (COL8A1); bone gamma-carboxyglutamate protein 3 (BGLAP-RS1); matrix Gla protein (MGP); matrix metallopeptidase 13 (MMP13); thrombospondin 1 (THBS1); bone gamma carboxyglutamate protein (BGLAP1); bone gamma-carboxyglutamate protein 2 (BGLAP2); actin, alpha 2, smooth muscle, aorta (ACTA2) and thymosin beta 4, X-linked (TMSB4X).

In some instances, a useful regulatory sequence may be a regulatory sequence that is upregulated by MYC. In some instances, regulatory sequences upregulated by MYC may include what are referred to herein as “Class II promoters” or the regulatory sequence of what is referred to herein as a “Class II gene”. Useful regulatory sequences that are upregulated by MYC, including but not limited to e.g., a regulatory sequence of one or more of the following MYC-upregulated genes: sphingomyelin phosphodiesterase acid like 3B (SMPDL3B); YdjC homolog (YDJC); serine hydroxymethyltransferase 1 (SHMT1); lipoprotein lipase (LPL); nucleoporin 210 (NUP210); PDZ and LIM domain 4 (PDLIM4); lysosomal protein transmembrane 5 (LAPTM5); MYB proto-oncogene like 2 (MYBL2); CD8b molecule (CD8B1); erythroid differentiation regulator 1 (ERDR1); inositol-3-phosphate synthase 1 (ISYNA1); T cell receptor beta, variable 13-2 (TCRB-V8.2); carbonic anhydrase 12 (CAR12); T cell receptor beta variable 1 (TRBV1); Thy-1 cell surface antigen (THY1); cyclin B1 interacting protein 1 (GM288); dipeptidyl peptidase 4 (DPP4); cathepsin E (CTSE); CD3g molecule (CD3G) and immunoglobulin heavy constant mu (IGH-6).

Useful MYC-responsive elements that are upregulated by MYC include Ebox containing elements. Ebox, or “enhancer box”, elements are DNA sequence response elements recognized and bound by transcription factors to regulate gene expression. The canonical Ebox consensus sequence is CANNTG, where N may be any nucleotide. Non-limiting exemplary Ebox sequences are often palindromic and include e.g., CACGTG and CAGCTG. Non-limiting examples of non-canonical Ebox sequences include e.g., CACGTT, CAGCTT and CACCTCGTGAC (SEQ ID NO:1). Myc, heterodimerized with MAX, may bind the Ebox sequence CAC(G/A)TG to activate transcription of target genes. In some instances, useful Ebox sequences bound by Myc may include the sequence CACGTG. In some instances, useful Ebox elements may include tandem repeats of Ebox sequences. The number of repeating units of sequence will be 2 or more and may vary any may include but are not limited to e.g., 2 or more, 3 or more, 4 or more, 5 or more, 2 to 10, 2 to 9, 2 to 8, 2 to 7, 2 to 6, 2 to 5, 2 to 4, 3 to 10, 3 to 9, 3 to 8, 3 to 7, 3 to 6, 3 to 5, 4 to 10, 4 to 9, 4 to 8, 4 to 7, 4 to 6, 5 to 10, 5 to 9, 5 to 8, 5 to 7, 10, 9, 8, 7, 6, 5, 4, 3, 2, and the like. In some instances, the number of repeating units of a tandem repeat may be referred to as having “x” number of repeating units, including e.g., a 5× Ebox tandem repeat contain five repeated Ebox sequences.

Individual units of a tandem repeat may or may not contain intervening sequence between the units, including where e.g., no intervening sequence is present between repeating units, 1 or more nucleotides are present between repeating units, 2 or more nucleotides are present between repeating units, 3 or more nucleotides are present between repeating units, 4 or more nucleotides are present between repeating units, 5 or more nucleotides are present between repeating units, 6 or more nucleotides are present between repeating units, etc. Where present, the number of intervening nucleotides between repeating units of a tandem repeat will generally, but not exclusively, be less than 20. In some instances, the nucleotides between repeating units, where present, may be referred to as a “spacer”.

Useful Ebox tandem repeats may be derived from regulatory sequences, such as mammalian (e.g., human, non-human primate, rodent, etc.) regulatory sequences, of genes that are regulated by Myc or may be modified forms of regulatory sequences that are bound by Myc or may be wholly or partially synthetically derived. In some instances, an Ebox tandem repeat may be employed as a substitute in a multi-MYC-reporter expression system for a regulatory sequence that is upregulated by MYC.

Detectable reporters suitable for use in the subject MYC reporters include, e.g., fluorescent proteins and luminescent proteins; enzymes that catalyze a reaction that generates a detectable signal as a product; epitope tags, surface markers, and the like. Detectable reporter proteins may be directly detected or indirectly detected. For example, where a fluorescent reporter is used, the fluorescence of the reporter may be directly detected. In some instances, where an epitope tag or a surface marker is used, the epitope tag or surface marker may be indirectly detected, e.g., through the use of a detectable binding agent that specifically binds the epitope tag or surface marker, e.g., a fluorescently labeled antibody that specifically binds the epitope tag or surface marker. In some instances, a reporter that is commonly indirectly detected, e.g., an epitope tag or surface marker, may be directed directly or a reporter that is commonly directly detected may be indirectly detected, e.g., through the use of a detectable antibody that specifically binds a fluorescent reporter.

Suitable fluorescent proteins include, but are not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilised EGFP (dEGFP), destabilised ECFP (dECFP), destabilised EYFP (dEYFP), mCFPm, Cerulean, T-Sapphire, CyPet, YPet, mKO, HcRed, t-HcRed, DsRed, DsRed2, DsRed-monomer, derivatives of DsRed and/or DsRed2, E2-Crimson, mKate2, mNeptune, mClover, mRuby, J-Red, dimer2, t-dimer2(12), mRFP1, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin. Other examples of fluorescent proteins include mHoneydew, mBanana, mOrange, dTomato, tdTomato, mTangerine, mStrawberry, mCherry, mGrape1, mRaspberry, mGrape2, mPlum (Shaner et al. (2005) Nat. Methods 2:905-909), and the like. Any of a variety of fluorescent and colored proteins from Anthozoan species, as described in, e.g., Matz et al. (1999) Nature Biotechnol. 17:969-973, is suitable for use. Suitable fluorescent proteins also include but are not limited to e.g., those described in Bajar et al. Nat Methods. 2016, 13(12):993-996; Bajar et al. Sci Rep. 2016, 6:20889 and Chu et al. Nat Methods. 2014, 11(5):572-578; the disclosures of which are incorporated herein by reference in their entirety.

Useful fluorescent proteins may in some instances include destabilized fluorescent proteins. Destabilized fluorescent proteins may be substituted for non-destabilized fluorescent proteins in essentially any context described herein and may find use, e.g., where increased reporter turnover is desired. In some instances, destabilized fluorescent proteins may be employed in vectors, cells, systems and methods of the present disclosure where a negative change in expression of the fluorescent protein is a possible outcome. For example, in some instances, a destabilized fluorescent protein may be employed, e.g., operably linked to a Class I regulatory sequence, where the presence of MYC inhibits the expression of the destabilized fluorescent protein. In some instances, a destabilized fluorescent protein may be employed where inhibition of MYC, e.g., through the application of a MYC inhibitor (MYCi), results in downregulation of the fluorescent reporter. These examples, should not be considered to be limiting and destabilized fluorescent proteins may be employed in essentially any appropriate context, including but not limited to e.g., where sequence encoding the destabilized fluorescent protein is operably linked to a Class I promoter or a Class II promoter, including where a Class I promoter is operably linked to a first destabilized fluorescent protein and a Class II promoter is operably linked to a second destabilized fluorescent protein.

Useful destabilized fluorescent proteins will vary may include those proteins that have been destabilized by any convenient and appropriate means. For example, in some instances, useful destabilized fluorescent proteins include those containing a PEST sequence. A PEST sequences is a peptide sequence that has a short intracellular half-life and is rich in proline (P), glutamic acid (E), serine (S), and threonine (T). Useful PEST sequences include wild-type and mutated PEST sequences, including e.g., the PEST containing mouse ornithine decarboxylase (MODC) sequence and derivatives thereof. Other methods of generating destabilized versions of fluorescent proteins may be employed, including but not limited to e.g., those employing a ubiquitin fusion strategy. Non-limiting examples of destabilized fluorescent proteins include but are not limited to e.g., PEST destabilized GFP (see e.g., Li et al., J Biol Chem. (1998) 273(52):34970-5; the disclosure of which is incorporated herein by reference in its entirety), Anthozoan destabilized fluorescent proteins (including e.g., those commercially available from Takara Bio USA, such as HcRed1-DR, DsRed-Express-DR, ZsGreen1-DR, etc.), N-degron destabilized GFP (see e.g., Houser et al., Yeast. (2012) 29(12):519-30; the disclosure of which is incorporated herein by reference in its entirety), and the like.

Suitable enzymes include, but are not limited to, horse radish peroxidase (HRP), alkaline phosphatase (AP), beta-galactosidase (GAL), glucose-6-phosphate dehydrogenase, beta-N-acetylglucosaminidase, β-glucuronidase, invertase, Xanthine Oxidase, firefly luciferase, renilla luciferase, glucose oxidase (GO), and the like.

In some instances, the subject MYC-reporters may be present in an expression vector and, as such, may be referred to as a MYC-reporter expression vector. The subject MYC-reporter expression vectors may include a single MYC-reporter containing a single MYC responsive gene regulatory element or may include multiple MYC-reporters (e.g., 2, 3, 4, 5 or more) each containing at least one MYC responsive gene regulatory element. In some instances, multi-MYC-reporter expression vectors may include a first reporter that is upregulated by MYC and a second reporter that is downregulated by MYC. In some instances, multi-MYC-reporter expression vectors any combination of MYC responsive gene regulatory elements, including e.g., those described herein may be employed.

In some instances, the MYC reporters and/or expression vectors may include a nucleotide sequence encoding a detectable structural protein. Detectable structural proteins may vary and may provide for analysis of cell morphology or morphological analysis of one or more components of a cell (e.g., nucleus, cytoplasm, etc.). In some embodiments, detectable structural proteins may include a structural protein (e.g., an actin, a tubulin, etc.) fused to a detectable protein (e.g., a fluorescent/luminescent protein), sometimes referred to as a “tagged structural protein”, wherein protein tagging may referred to the expression of a detectable protein (e.g., a fluorescent/luminescent protein) fused (e.g., with or without a linker) to the protein of interest. Any convenient and appropriate combination of structural protein and detectable protein may be employed.

One or more MYC reporters and/or one or more expression vectors containing MYC reporter(s) may be present in a cell, including e.g., a neoplastic cell or a cancer cell. A cell containing a MYC reporter and/or expression vector may be a cell of an isolated and/or cultured cell line or a cell present in an multicellular organism (such as an animal (e.g., a transgenic animal), e.g., a mammal, such as e.g., a rodent (e.g., a mouse, a rat, etc.), a non-human primate, etc.). Such cells may include a MYC expression construct (e.g., expressing a heterologous MYC), including e.g., an inducible MYC, a constitutive MYC, or the like. MYC reporters may be present extrachromosomally or may be integrated into a chromosome of a host cell genome.

As summarized above, also provided are cell lines containing one or more MYC reporters for use in various methods, including e.g., screening methods. The subject cell lines include neoplastic cell lines (e.g., tumor cell lines, cancer cell lines, etc.). In some instances, the subject cell lines are neoplasms derived from a mammalian neoplasia such as e.g., a human neoplasia, a mouse neoplasia, a rat neoplasia, a primate neoplasia, or the like. Any convenient cancer cell may be employed including but not limited to e.g., a hepatocellular carcinoma cell, a T-cell acute lymphoblastic leukemia cell, an osteosarcoma cell, a renal cell carcinoma cell, a lung cancer cell, or the like. Any convenient cancer cell line may be employed including but not limited to e.g., a hepatocellular carcinoma cell line, a T-cell acute lymphoblastic leukemia cell line, an osteosarcoma cell line, a renal cell carcinoma cell line, a lung cancer cell line, or the like.

Any cell or cell line of a cancer may be adapted to include a MYC reporter of the present disclosure. Cancers of interest include but are not limited to e.g., Acute Lymphoblastic Leukemia (ALL), Acute Myeloid Leukemia (AML), Adrenocortical Carcinoma, AIDS-Related Cancers (e.g., Kaposi Sarcoma, Lymphoma, etc.), Anal Cancer, Appendix Cancer, Astrocytomas, Atypical Teratoid/Rhabdoid Tumor, Basal Cell Carcinoma, Bile Duct Cancer (Extrahepatic), Bladder Cancer, Bone Cancer (e.g., Ewing Sarcoma, Osteosarcoma and Malignant Fibrous Histiocytoma, etc.), Brain Stem Glioma, Brain Tumors (e.g., Astrocytomas, Central Nervous System Embryonal Tumors, Central Nervous System Germ Cell Tumors, Craniopharyngioma, Ependymoma, etc.), Breast Cancer (e.g., female breast cancer, male breast cancer, childhood breast cancer, etc.), Bronchial Tumors, Burkitt Lymphoma, Carcinoid Tumor (e.g., Childhood, Gastrointestinal, etc.), Carcinoma of Unknown Primary, Cardiac (Heart) Tumors, Central Nervous System (e.g., Atypical Teratoid/Rhabdoid Tumor, Embryonal Tumors, Germ Cell Tumor, Lymphoma, etc.), Cervical Cancer, Childhood Cancers, Chordoma, Chronic Lymphocytic Leukemia (CLL), Chronic Myelogenous Leukemia (CML), Chronic Myeloproliferative Neoplasms, Colon Cancer, Colorectal Cancer, Craniopharyngioma, Cutaneous T-Cell Lymphoma, Duct (e.g., Bile Duct, Extrahepatic, etc.), Ductal Carcinoma In Situ (DCIS), Embryonal Tumors, Endometrial Cancer, Ependymoma, Esophageal Cancer, Esthesioneuroblastoma, Ewing Sarcoma, Extracranial Germ Cell Tumor, Extragonadal Germ Cell Tumor, Extrahepatic Bile Duct Cancer, Eye Cancer (e.g., Intraocular Melanoma, Retinoblastoma, etc.), Fibrous Histiocytoma of Bone (e.g., Malignant, Osteosarcoma, ect.), Gallbladder Cancer, Gastric (Stomach) Cancer, Gastrointestinal Carcinoid Tumor, Gastrointestinal Stromal Tumors (GIST), Germ Cell Tumor (e.g., Extracranial, Extragonadal, Ovarian, Testicular, etc.), Gestational Trophoblastic Disease, Glioma, Hairy Cell Leukemia, Head and Neck Cancer, Heart Cancer, Hepatocellular (Liver) Cancer, Histiocytosis (e.g., Langerhans Cell, etc.), Hodgkin Lymphoma, Hypopharyngeal Cancer, Intraocular Melanoma, Islet Cell Tumors (e.g., Pancreatic Neuroendocrine Tumors, etc.), Kaposi Sarcoma, Kidney Cancer (e.g., Renal Cell, Wilms Tumor, Childhood Kidney Tumors, etc.), Langerhans Cell Histiocytosis, Laryngeal Cancer, Leukemia (e.g., Acute Lymphoblastic (ALL), Acute Myeloid (AML), Chronic Lymphocytic (CLL), Chronic Myelogenous (CML), Hairy Cell, etc.), Lip and Oral Cavity Cancer, Liver Cancer (Primary), Lobular Carcinoma In Situ (LCIS), Lung Cancer (e.g., Non-Small Cell, Small Cell, etc.), Lymphoma (e.g., AIDS-Related, Burkitt, Cutaneous T-Cell, Hodgkin, Non-Hodgkin, Primary Central Nervous System (CNS), etc.), Macroglobulinemia (e.g., Waldenström, etc.), Male Breast Cancer, Malignant Fibrous Histiocytoma of Bone and Osteosarcoma, Melanoma, Merkel Cell Carcinoma, Mesothelioma, Metastatic Squamous Neck Cancer with Occult Primary, Midline Tract Carcinoma Involving NUT Gene, Mouth Cancer, Multiple Endocrine Neoplasia Syndromes, Multiple Myeloma/Plasma Cell Neoplasm, Mycosis Fungoides, Myelodysplastic Syndromes, Myelodysplastic/Myeloproliferative Neoplasms, Myelogenous Leukemia (e.g., Chronic (CML), etc.), Myeloid Leukemia (e.g., Acute (AML), etc.), Myeloproliferative Neoplasms (e.g., Chronic, etc.), Nasal Cavity and Paranasal Sinus Cancer, Nasopharyngeal Cancer, Neuroblastoma, Non-Hodgkin Lymphoma, Non-Small Cell Lung Cancer, Oral Cancer, Oral Cavity Cancer (e.g., Lip, etc.), Oropharyngeal Cancer, Osteosarcoma and Malignant Fibrous Histiocytoma of Bone, Ovarian Cancer (e.g., Epithelial, Germ Cell Tumor, Low Malignant Potential Tumor, etc.), Pancreatic Cancer, Pancreatic Neuroendocrine Tumors (Islet Cell Tumors), Papillomatosis, Paraganglioma, Paranasal Sinus and Nasal Cavity Cancer, Parathyroid Cancer, Penile Cancer, Pharyngeal Cancer, Pheochromocytoma, Pituitary Tumor, Pleuropulmonary Blastoma, Primary Central Nervous System (CNS) Lymphoma, Prostate Cancer, Rectal Cancer, Renal Cell (Kidney) Cancer, Renal Pelvis and Ureter, Transitional Cell Cancer, Retinoblastoma, Rhabdomyosarcoma, Salivary Gland Cancer, Sarcoma (e.g., Ewing, Kaposi, Osteosarcoma, Rhabdomyosarcoma, Soft Tissue, Uterine, etc.), Sézary Syndrome, Skin Cancer (e.g., Childhood, Melanoma, Merkel Cell Carcinoma, Nonmelanoma, etc.), Small Cell Lung Cancer, Small Intestine Cancer, Soft Tissue Sarcoma, Squamous Cell Carcinoma, Squamous Neck Cancer (e.g., with Occult Primary, Metastatic, etc.), Stomach (Gastric) Cancer, T-Cell Lymphoma, Testicular Cancer, Throat Cancer, Thymoma and Thymic Carcinoma, Thyroid Cancer, Transitional Cell Cancer of the Renal Pelvis and Ureter, Ureter and Renal Pelvis Cancer, Urethral Cancer, Uterine Cancer (e.g., Endometrial, etc.), Uterine Sarcoma, Vaginal Cancer, Vulvar Cancer, Waldenström Macroglobulinemia, Wilms Tumor, and the like.

The subject cell lines and/or reporters may be provided as part of a kit for use of the cells and/or reporters in a screening method. Such kits may include any combination of components (e.g., reagents, cell lines, etc.) for performing the subject methods, such as e.g., methods of screening a candidate agent for MYC repression).

In addition to the above components, the subject kits and/or cell lines and/or reporters may further include (in certain embodiments) instructions for practicing the subject screening methods. These instructions may be present in the subject kits or provided with the subject cell lines and/or reporters in a variety of forms, one or more of which may be present in the kit or provided with the subject cell lines and/or reporters. One form in which these instructions may be present is as printed information on a suitable medium or substrate, e.g., a piece or pieces of paper on which the information is printed, in the packaging of the kit or cell line(s), in a package insert, and the like. Yet another form of these instructions is a computer readable medium, e.g., diskette, compact disk (CD), flash drive, and the like, on which the information has been recorded. Yet another form of these instructions that may be present is a website address which may be used via the internet to access the information at a removed site.

### Methods

As summarized above, the present disclosure also provides methods of screening. The subject screening methods may be employed to identify whether a candidate agent is an agent that represses or otherwise antagonizes MYC and/or MYC signaling. Such screening methods may be generally referred to as methods for screening a candidate agent for MYC repression and the like.

The subject methods may include contacting a MYC-driven cancer cell, that includes one or more MYC reporters (or expression vector(s)), with a candidate agent. As used herein, the term “MYC driven” as it refers to cells (e.g., cancer cells) will generally describe a cell having an elevated level of MYC and/or overexpressing MYC, including e.g., where MYC is elevated and/or overexpressed due to genetic modification of the cell or where MYC is elevated and/or overexpressed as a result of a cancer pathology (including e.g., a genetic lesion associated with the cancer) and the like. Any convenient mode of contacting may be employed depending on the particular method of screening employed including e.g., adding the agent to a culture medium of a cell culture, administering the agent to a multicellular organism host of the cell, etc. Contacting may be performed in vitro, in vivo, or the like.

The subject methods may include detecting a change in the expression of at least one detectable reporter of the MYC-reporter expression vector in the MYC-driven cancer cell. Such detecting may be performed at any convenient time, including e.g., during or after the cell is contacted with the candidate agent. Such detecting may be performed using any convenient means depending on the detectable reporter employed. For example, in some instances a fluorescent/luminescent reporter may be employed and the detecting may include detecting a change in fluorescence/luminescence (e.g., a change in fluorescence/luminescent intensity) by any convenient means (e.g., a luminometer, a fluorometer, fluorescent microscopy, flow cytometry (e.g., FACS), etc.).

A MYC reporter employed in the subject methods may include one or more detectable reporters, where each detectable reporter is operably linked to a regulatory sequence that is up or down regulated by MYC. In some embodiments, the one or more regulatory sequences may be a regulatory sequence of a MYC-responsive gene selected from the group consisting of: expressed sequence AI593442 (AI593442); 3′-phosphoadenosine 5′-phosphosulfate synthase 2 (PAPSS2); proline and arginine rich end leucine rich repeat protein (PRELP); TNF receptor superfamily member 11 b (TNFRSF11B); ribonuclease A family member 4 (RNASE4); ADAM metallopeptidase with thrombospondin type 1 motif 2 (ADAMTS2); guanylate binding protein 2 (GBP2); S100 calcium binding protein A13 (S100A13); porcupine homolog (PORCN); FAM20A, golgi associated secretory pathway pseudokinase (FAM20A); actin, gamma 2, smooth muscle, enteric (ACTG2); collagen type VIII alpha 1 chain (COL8A1); bone gamma-carboxyglutamate protein 3 (BGLAP-RS1); matrix Gla protein (MGP); matrix metallopeptidase 13 (MMP13); thrombospondin 1 (THBS1); bone gamma carboxyglutamate protein (BGLAP1); bone gamma-carboxyglutamate protein 2 (BGLAP2); actin, alpha 2, smooth muscle, aorta (ACTA2); thymosin beta 4, X-linked (TMSB4X); sphingomyelin phosphodiesterase acid like 3B (SMPDL3B); YdjC homolog (YDJC); serine hydroxymethyltransferase 1 (SHMT1); lipoprotein lipase (LPL); nucleoporin 210 (NUP210); PDZ and LIM domain 4 (PDLIM4); lysosomal protein transmembrane 5 (LAPTM5); MYB proto-oncogene like 2 (MYBL2); CD8b molecule (CD8B1); erythroid differentiation regulator 1 (ERDR1); inositol-3-phosphate synthase 1 (ISYNA1); T cell receptor beta, variable 13-2 (TCRB-V8.2); carbonic anhydrase 12 (CAR12); T cell receptor beta variable 1 (TRBV1); Thy-1 cell surface antigen (THY1); cyclin B1 interacting protein 1 (GM288); dipeptidyl peptidase 4 (DPP4); cathepsin E (CTSE); CD3g molecule (CD3G) and immunoglobulin heavy constant mu (IGH-6).

Useful regulatory sequences include AI593442 (also known as expressed sequence AI593442) regulatory sequences (e.g., a AI593442 promoter), including those regulating the expression of a gene encoding RefSeq NP_001273570.1 (SEQ ID NO:2) (Uniprot ID Q32M26, SEQ ID NO:41) and/or mouse/human orthologs/homologs thereof. In some instances, useful AI593442 regulatory sequences may include sequence upstream of a AI593442 start site including but not limited to e.g., sequence more than 5 kb or 5 kb or less, 4 kb or less, 3 kb or less, 2 kb or less, or 1 kb or less upstream from a AI593442 start site, including but not limited to e.g., the start site of UniGene ID Mm.335160.

Useful regulatory sequences include C11orf87 (also known as Uncharacterized protein C11orf87) regulatory sequences (e.g., a C11orf87 promoter), including those regulating the expression of a gene encoding RefSeq NP_997528.2 (SEQ ID NO:3) (Uniprot ID Q6NUJ2, SEQ ID NO:42) and/or mouse/human orthologs/homologs thereof. In some instances, useful C11orf87 regulatory sequences may include sequence upstream of a C11orf87 start site including but not limited to e.g., sequence more than 5 kb or 5 kb or less, 4 kb or less, 3 kb or less, 2 kb or less, or 1 kb or less upstream from a C11orf87 start site, including but not limited to e.g., the start site of UniGene ID Hs.172982.

Useful regulatory sequences include PAPSS2 (also known as ATPSK2) regulatory sequences (e.g., a PAPSS2 promoter), including those regulating the expression of a gene encoding RefSeq NP_001015880.1 (SEQ ID NO:4) (Uniprot ID 095340, SEQ ID NO:43) and/or mouse/human orthologs/homologs thereof. In some instances, useful PAPSS2 regulatory sequences may include sequence upstream of a PAPSS2 start site including but not limited to e.g., sequence more than 5 kb or 5 kb or less, 4 kb or less, 3 kb or less, 2 kb or less, or 1 kb or less upstream from a PAPSS2 start site, including but not limited to e.g., the start site of UniGene ID Hs.524491.

Useful regulatory sequences include PRELP (also known as SLRR2A) regulatory sequences (e.g., a PRELP promoter), including those regulating the expression of a gene encoding RefSeq NP_002716.1 (SEQ ID NO:5) (Uniprot ID P51888, SEQ ID NO:44) and/or mouse/human orthologs/homologs thereof. In some instances, useful PRELP regulatory sequences may include sequence upstream of a PRELP start site including but not limited to e.g., sequence more than 5 kb or 5 kb or less, 4 kb or less, 3 kb or less, 2 kb or less, or 1 kb or less upstream from a PRELP start site, including but not limited to e.g., the start site of UniGene ID Hs.632481.

Useful regulatory sequences include TNFRSF11B (also known as OCIF and OPG) regulatory sequences (e.g., a TNFRSF11B promoter), including those regulating the expression of a gene encoding RefSeq NP_002537.3 (SEQ ID NO:6) (Uniprot ID 000300, SEQ ID NO:45) and/or mouse/human orthologs/homologs thereof. In some instances, useful TNFRSF11B regulatory sequences may include sequence upstream of a TNFRSF11B start site including but not limited to e.g., sequence more than 5 kb or 5 kb or less, 4 kb or less, 3 kb or less, 2 kb or less, or 1 kb or less upstream from a TNFRSF11B start site, including but not limited to e.g., the start site of UniGene ID Hs.81791.

Useful regulatory sequences include RNASE4 (also known as RNS4) regulatory sequences (e.g., a RNASE4 promoter), including those regulating the expression of a gene encoding RefSeq NP_001269121.1 (SEQ ID NO:7) (Uniprot ID P34096, SEQ ID NO:46) and/or mouse/human orthologs/homologs thereof. In some instances, useful RNASE4 regulatory sequences may include sequence upstream of a RNASE4 start site including but not limited to e.g., sequence more than 5 kb or 5 kb or less, 4 kb or less, 3 kb or less, 2 kb or less, or 1 kb or less upstream from a RNASE4 start site, including but not limited to e.g., the start site of UniGene ID Hs.283749.

Useful regulatory sequences include ADAMTS2 (also known as PCINP and PCPNI) regulatory sequences (e.g., a ADAMTS2 promoter), including those regulating the expression of a gene encoding RefSeq NP_055059.2 (SEQ ID NO:8) (Uniprot ID 095450, SEQ ID NO:47) and/or mouse/human orthologs/homologs thereof. In some instances, useful ADAMTS2 regulatory sequences may include sequence upstream of a ADAMTS2 start site including but not limited to e.g., sequence more than 5 kb or 5 kb or less, 4 kb or less, 3 kb or less, 2 kb or less, or 1 kb or less upstream from a ADAMTS2 start site, including but not limited to e.g., the start site of UniGene ID Hs.23871.

Useful regulatory sequences include GBP2 (also known as Guanylate-binding protein 2) regulatory sequences (e.g., a GBP2 promoter), including those regulating the expression of a gene encoding RefSeq NP_004111.2 (SEQ ID NO:9) (Uniprot ID P32456, SEQ ID NO:48) and/or mouse/human orthologs/homologs thereof. In some instances, useful GBP2 regulatory sequences may include sequence upstream of a GBP2 start site including but not limited to e.g., sequence more than 5 kb or 5 kb or less, 4 kb or less, 3 kb or less, 2 kb or less, or 1 kb or less upstream from a GBP2 start site, including but not limited to e.g., the start site of UniGene ID Hs.386567.

Useful regulatory sequences include S100A13 (also known as S100 calcium-binding protein A13) regulatory sequences (e.g., a S100A13 promoter), including those regulating the expression of a gene encoding RefSeq NP_001019381.1 (SEQ ID NO:10) (Uniprot ID Q99584, SEQ ID NO:49) and/or mouse/human orthologs/homologs thereof. In some instances, useful S100A13 regulatory sequences may include sequence upstream of a S100A13 start site including but not limited to e.g., sequence more than 5 kb or 5 kb or less, 4 kb or less, 3 kb or less, 2 kb or less, or 1 kb or less upstream from a S100A13 start site, including but not limited to e.g., the start site of UniGene ID Hs.516505.

Useful regulatory sequences include PORCN (also known as MG61, PORC and PPN) regulatory sequences (e.g., a PORCN promoter), including those regulating the expression of a gene encoding RefSeq NP_001269096.1 (SEQ ID NO:11) (Uniprot ID Q9H237, SEQ ID NO:50) and/or mouse/human orthologs/homologs thereof. In some instances, useful PORCN regulatory sequences may include sequence upstream of a PORCN start site including but not limited to e.g., sequence more than 5 kb or 5 kb or less, 4 kb or less, 3 kb or less, 2 kb or less, or 1 kb or less upstream from a PORCN start site, including but not limited to e.g., the start site of UniGene ID Hs.386453.

Useful regulatory sequences include FAM20A (also known as UNQ9388/PRO34279) regulatory sequences (e.g., a FAM20A promoter), including those regulating the expression of a gene encoding RefSeq NP_001230675.1 (SEQ ID NO:12) (Uniprot ID Q96MK3, SEQ ID NO:51) and/or mouse/human orthologs/homologs thereof. In some instances, useful FAM20A regulatory sequences may include sequence upstream of a FAM20A start site including but not limited to e.g., sequence more than 5 kb or 5 kb or less, 4 kb or less, 3 kb or less, 2 kb or less, or 1 kb or less upstream from a FAM20A start site, including but not limited to e.g., the start site of UniGene ID Hs.268874.

Useful regulatory sequences include ACTG2 (also known as ACTA3, ACTL3 and ACTSG) regulatory sequences (e.g., a ACTG2 promoter), including those regulating the expression of a gene encoding RefSeq NP_001186822.1 (SEQ ID NO:13) (Uniprot ID P63267, SEQ ID NO:52) and/or mouse/human orthologs/homologs thereof. In some instances, useful ACTG2 regulatory sequences may include sequence upstream of a ACTG2 start site including but not limited to e.g., sequence more than 5 kb or 5 kb or less, 4 kb or less, 3 kb or less, 2 kb or less, or 1 kb or less upstream from a ACTG2 start site, including but not limited to e.g., the start site of UniGene ID Hs.516105.

Useful regulatory sequences include COL8A1 (also known as C3orf7) regulatory sequences (e.g., a COL8A1 promoter), including those regulating the expression of a gene encoding RefSeq NP_001841.2 (SEQ ID NO:14) (Uniprot ID P27658, SEQ ID NO:53) and/or mouse/human orthologs/homologs thereof. In some instances, useful COL8A1 regulatory sequences may include sequence upstream of a COL8A1 start site including but not limited to e.g., sequence more than 5 kb or 5 kb or less, 4 kb or less, 3 kb or less, 2 kb or less, or 1 kb or less upstream from a COL8A1 start site, including but not limited to e.g., the start site of UniGene ID Hs.654548.

Useful regulatory sequences include BGLAP-RS1 (also known as Bglap3 and Bglap-rs1) regulatory sequences (e.g., a BGLAP-RS1 promoter), including those regulating the expression of a gene encoding RefSeq NP_001292377.1 (SEQ ID NO:15) (Uniprot ID P54615, SEQ ID NO:54) and/or mouse/human orthologs/homologs thereof. In some instances, useful BGLAP-RS1 regulatory sequences may include sequence upstream of a BGLAP-RS1 start site including but not limited to e.g., sequence more than 5 kb or 5 kb or less, 4 kb or less, 3 kb or less, 2 kb or less, or 1 kb or less upstream from a BGLAP-RS1 start site, including but not limited to e.g., the start site of UniGene ID Mm.482191.

Useful regulatory sequences include MGP (also known as MGLAP and GIG36) regulatory sequences (e.g., a MGP promoter), including those regulating the expression of a gene encoding RefSeq NP_000891.2 (SEQ ID NO:16) (Uniprot ID P08493, SEQ ID NO:55) and/or mouse/human orthologs/homologs thereof. In some instances, useful MGP regulatory sequences may include sequence upstream of a MGP start site including but not limited to e.g., sequence more than 5 kb or 5 kb or less, 4 kb or less, 3 kb or less, 2 kb or less, or 1 kb or less upstream from a MGP start site, including but not limited to e.g., the start site of UniGene ID Hs.365706.

Useful regulatory sequences include MMP13 (also known as Collagenase 3) regulatory sequences (e.g., a MMP13 promoter), including those regulating the expression of a gene encoding RefSeq NP_002418.1 (SEQ ID NO:17) (Uniprot ID P45452, SEQ ID NO:56) and/or mouse/human orthologs/homologs thereof. In some instances, useful MMP13 regulatory sequences may include sequence upstream of a MMP13 start site including but not limited to e.g., sequence more than 5 kb or 5 kb or less, 4 kb or less, 3 kb or less, 2 kb or less, or 1 kb or less upstream from a MMP13 start site, including but not limited to e.g., the start site of UniGene ID Hs.2936.

Useful regulatory sequences include THBS1 (also known as TSP and TSP1) regulatory sequences (e.g., a THBS1 promoter), including those regulating the expression of a gene encoding RefSeq NP_003237.2 (SEQ ID NO:18) (Uniprot ID P07996, SEQ ID NO:57) and/or mouse/human orthologs/homologs thereof. In some instances, useful THBS1 regulatory sequences may include sequence upstream of a THBS1 start site including but not limited to e.g., sequence more than 5 kb or 5 kb or less, 4 kb or less, 3 kb or less, 2 kb or less, or 1 kb or less upstream from a THBS1 start site, including but not limited to e.g., the start site of UniGene ID Hs.164226.

Useful regulatory sequences include BGLAP1 (also known as mouse Osteocalcin) regulatory sequences (e.g., a BGLAP1 promoter), including those regulating the expression of a gene encoding RefSeq NP_031567.1 (SEQ ID NO:19) (Uniprot ID P86546, SEQ ID NO:58) and/or mouse/human orthologs/homologs thereof. In some instances, useful BGLAP1 regulatory sequences may include sequence upstream of a BGLAP1 start site including but not limited to e.g., sequence more than 5 kb or 5 kb or less, 4 kb or less, 3 kb or less, 2 kb or less, or 1 kb or less upstream from a BGLAP1 start site, including but not limited to e.g., the start site of UniGene ID Mm.389459.

Useful regulatory sequences include BGLAP1 (also known as human Osteocalcin) regulatory sequences (e.g., a BGLAP1 promoter), including those regulating the expression of a gene encoding RefSeq NP_954642.1 (SEQ ID NO:20) (Uniprot ID P02818, SEQ ID NO:59) and/or mouse/human orthologs/homologs thereof. In some instances, useful BGLAP1 regulatory sequences may include sequence upstream of a BGLAP1 start site including but not limited to e.g., sequence more than 5 kb or 5 kb or less, 4 kb or less, 3 kb or less, 2 kb or less, or 1 kb or less upstream from a BGLAP1 start site, including but not limited to e.g., the start site of UniGene ID Hs.654541.

Useful regulatory sequences include BGLAP2 (also known as Osteocalcin-2) regulatory sequences (e.g., a BGLAP2 promoter), including those regulating the expression of a gene encoding RefSeq NP_001027469.2 (SEQ ID NO:21) (Uniprot ID P86547, SEQ ID NO:60) and/or mouse/human orthologs/homologs thereof. In some instances, useful BGLAP2 regulatory sequences may include sequence upstream of a BGLAP2 start site including but not limited to e.g., sequence more than 5 kb or 5 kb or less, 4 kb or less, 3 kb or less, 2 kb or less, or 1 kb or less upstream from a BGLAP2 start site, including but not limited to e.g., the start site of UniGene ID Mm.425160.

Useful regulatory sequences include ACTA2 (also known as ACTSA, ACTVS and GIG46) regulatory sequences (e.g., a ACTA2 promoter), including those regulating the expression of a gene encoding RefSeq NP_001135417.1 (SEQ ID NO:22) (Uniprot ID P62736, SEQ ID NO:61) and/or mouse/human orthologs/homologs thereof. In some instances, useful ACTA2 regulatory sequences may include sequence upstream of a ACTA2 start site including but not limited to e.g., sequence more than 5 kb or 5 kb or less, 4 kb or less, 3 kb or less, 2 kb or less, or 1 kb or less upstream from a ACTA2 start site, including but not limited to e.g., the start site of UniGene ID Hs.500483.

Useful regulatory sequences include TMSB4X (also known as TB4X, THYB4 and TMSB4) regulatory sequences (e.g., a TMSB4X promoter), including those regulating the expression of a gene encoding RefSeq NP_066932.1 (SEQ ID NO:23) (Uniprot ID P62328, SEQ ID NO:62) and/or mouse/human orthologs/homologs thereof. In some instances, useful TMSB4X regulatory sequences may include sequence upstream of a TMSB4X start site including but not limited to e.g., sequence more than 5 kb or 5 kb or less, 4 kb or less, 3 kb or less, 2 kb or less, or 1 kb or less upstream from a TMSB4X start site, including but not limited to e.g., the start site of UniGene ID Hs.437277.

Useful regulatory sequences include SMPDL3B (also known as ASML3B and ASMLPD) regulatory sequences (e.g., a SMPDL3B promoter), including those regulating the expression of a gene encoding RefSeq NP_001009568.1 (SEQ ID NO:24) (Uniprot ID Q92485, SEQ ID NO:63) and/or mouse/human orthologs/homologs thereof. In some instances, useful SMPDL3B regulatory sequences may include sequence upstream of a SMPDL3B start site including but not limited to e.g., sequence more than 5 kb or 5 kb or less, 4 kb or less, 3 kb or less, 2 kb or less, or 1 kb or less upstream from a SMPDL3B start site, including but not limited to e.g., the start site of UniGene ID Hs.123659.

Useful regulatory sequences include YDJC (also known as Carbohydrate deacetylase) regulatory sequences (e.g., a YDJC promoter), including those regulating the expression of a gene encoding RefSeq NP_001017964.1 (SEQ ID NO:25) (Uniprot ID A8MPS7, SEQ ID NO:64) and/or mouse/human orthologs/homologs thereof. In some instances, useful YDJC regulatory sequences may include sequence upstream of a YDJC start site including but not limited to e.g., sequence more than 5 kb or 5 kb or less, 4 kb or less, 3 kb or less, 2 kb or less, or 1 kb or less upstream from a YDJC start site, including but not limited to e.g., the start site of UniGene ID Hs.355952.

Useful regulatory sequences include SHMT1 (also known as Serine hydroxymethyltransferase, cytosolic) regulatory sequences (e.g., a SHMT1 promoter), including those regulating the expression of a gene encoding RefSeq NP_001268715.1 (SEQ ID NO:26) (Uniprot ID P34896, SEQ ID NO:65) and/or mouse/human orthologs/homologs thereof. In some instances, useful SHMT1 regulatory sequences may include sequence upstream of a SHMT1 start site including but not limited to e.g., sequence more than 5 kb or 5 kb or less, 4 kb or less, 3 kb or less, 2 kb or less, or 1 kb or less upstream from a SHMT1 start site, including but not limited to e.g., the start site of UniGene ID Hs.513987.

Useful regulatory sequences include LPL (also known as LIPD) regulatory sequences (e.g., a LPL promoter), including those regulating the expression of a gene encoding RefSeq NP_000228.1 (SEQ ID NO:27) (Uniprot ID P06858, SEQ ID NO:66) and/or mouse/human orthologs/homologs thereof. In some instances, useful LPL regulatory sequences may include sequence upstream of a LPL start site including but not limited to e.g., sequence more than 5 kb or 5 kb or less, 4 kb or less, 3 kb or less, 2 kb or less, or 1 kb or less upstream from a LPL start site, including but not limited to e.g., the start site of UniGene ID Hs.180878.

Useful regulatory sequences include NUP210 (also known as KIAA0906 and PSEC0245) regulatory sequences (e.g., a NUP210 promoter), including those regulating the expression of a gene encoding RefSeq NP_079199.2 (SEQ ID NO:28) (Uniprot ID Q8TEM1, SEQ ID NO:67) and/or mouse/human orthologs/homologs thereof. In some instances, useful NUP210 regulatory sequences may include sequence upstream of a NUP210 start site including but not limited to e.g., sequence more than 5 kb or 5 kb or less, 4 kb or less, 3 kb or less, 2 kb or less, or 1 kb or less upstream from a NUP210 start site, including but not limited to e.g., the start site of UniGene ID Hs.475525.

Useful regulatory sequences include PDLIM4 (also known as RIL) regulatory sequences (e.g., a PDLIM4 promoter), including those regulating the expression of a gene encoding RefSeq NP_001124499.1 (SEQ ID NO:29) (Uniprot ID P50479, SEQ ID NO:68) and/or mouse/human orthologs/homologs thereof. In some instances, useful PDLIM4 regulatory sequences may include sequence upstream of a PDLIM4 start site including but not limited to e.g., sequence more than 5 kb or 5 kb or less, 4 kb or less, 3 kb or less, 2 kb or less, or 1 kb or less upstream from a PDLIM4 start site, including but not limited to e.g., the start site of UniGene ID Hs.424312.

Useful regulatory sequences include LAPTM5 (also known as KIAA0085) regulatory sequences (e.g., a LAPTM5 promoter), including those regulating the expression of a gene encoding RefSeq NP_006753.1 (SEQ ID NO:30) (Uniprot ID Q13571, SEQ ID NO:69) and/or mouse/human orthologs/homologs thereof. In some instances, useful LAPTM5 regulatory sequences may include sequence upstream of a LAPTM5 start site including but not limited to e.g., sequence more than 5 kb or 5 kb or less, 4 kb or less, 3 kb or less, 2 kb or less, or 1 kb or less upstream from a LAPTM5 start site, including but not limited to e.g., the start site of UniGene ID Hs.371021.

Useful regulatory sequences include MYBL2 (also known as BMYB) regulatory sequences (e.g., a MYBL2 promoter), including those regulating the expression of a gene encoding RefSeq NP_001265539.1 (SEQ ID NO:31) (Uniprot ID P10244, SEQ ID NO:70) and/or mouse/human orthologs/homologs thereof. In some instances, useful MYBL2 regulatory sequences may include sequence upstream of a MYBL2 start site including but not limited to e.g., sequence more than 5 kb or 5 kb or less, 4 kb or less, 3 kb or less, 2 kb or less, or 1 kb or less upstream from a MYBL2 start site, including but not limited to e.g., the start site of UniGene ID Hs.179718.

Useful regulatory sequences include CD8B1 (also known as CD8B) regulatory sequences (e.g., a CD8B1 promoter), including those regulating the expression of a gene encoding RefSeq NP_001171571.1 (SEQ ID NO:32) (Uniprot ID P10966, SEQ ID NO:71) and/or mouse/human orthologs/homologs thereof. In some instances, useful CD8B1 regulatory sequences may include sequence upstream of a CD8B1 start site including but not limited to e.g., sequence more than 5 kb or 5 kb or less, 4 kb or less, 3 kb or less, 2 kb or less, or 1 kb or less upstream from a CD8B1 start site, including but not limited to e.g., the start site of UniGene ID Hs.405667.

Useful regulatory sequences include ERDR1 (also known as Erythroid differentiation regulator 1) regulatory sequences (e.g., a ERDR1 promoter), including those regulating the expression of a gene encoding RefSeq NP_579940.1 (SEQ ID NO:33) (Uniprot ID Q8VEL1, SEQ ID NO:72) and/or mouse/human orthologs/homologs thereof. In some instances, useful ERDR1 regulatory sequences may include sequence upstream of a ERDR1 start site including but not limited to e.g., sequence more than 5 kb or 5 kb or less, 4 kb or less, 3 kb or less, 2 kb or less, or 1 kb or less upstream from a ERDR1 start site, including but not limited to e.g., the start site of UniGene ID Mm.391385.

Useful regulatory sequences include ISYNA1 (also known as INO1) regulatory sequences (e.g., a ISYNA1 promoter), including those regulating the expression of a gene encoding RefSeq NP_001164409.1 (SEQ ID NO:34) (Uniprot ID Q9NPH2, SEQ ID NO:73) and/or mouse/human orthologs/homologs thereof. In some instances, useful ISYNA1 regulatory sequences may include sequence upstream of a ISYNA1 start site including but not limited to e.g., sequence more than 5 kb or 5 kb or less, 4 kb or less, 3 kb or less, 2 kb or less, or 1 kb or less upstream from a ISYNA1 start site, including but not limited to e.g., the start site of UniGene ID Hs.405873.

Useful regulatory sequences include TCRB-V8.2 (also known as TRAV13-2) regulatory sequences (e.g., a TCRB-V8.2 promoter), including those regulating the expression of a gene encoding Uniprot ID A0A0B4J235 (SEQ ID NO:74) and/or mouse/human orthologs/homologs thereof. In some instances, useful TCRB-V8.2 regulatory sequences may include sequence upstream of a TCRB-V8.2 start site including but not limited to e.g., sequence more than 5 kb or 5 kb or less, 4 kb or less, 3 kb or less, 2 kb or less, or 1 kb or less upstream from a TCRB-V8.2 start site, including but not limited to e.g., the start site of UniGene ID GenBank: AC243980.3.

Useful regulatory sequences include CAR12 (also known as Carbonate dehydratase XII) regulatory sequences (e.g., a CAR12 promoter), including those regulating the expression of a gene encoding RefSeq NP_001209.1 (SEQ ID NO:35) (Uniprot ID 043570, SEQ ID NO:75) and/or mouse/human orthologs/homologs thereof. In some instances, useful CAR12 regulatory sequences may include sequence upstream of a CAR12 start site including but not limited to e.g., sequence more than 5 kb or 5 kb or less, 4 kb or less, 3 kb or less, 2 kb or less, or 1 kb or less upstream from a CAR12 start site, including but not limited to e.g., the start site of UniGene ID Hs.210995.

Useful regulatory sequences include TRBV1 (also known as T cell receptor beta, variable 1) regulatory sequences (e.g., a TRBV1 promoter), including those regulating the expression of a gene encoding Uniprot ID A0A0B4J1G7 (SEQ ID NO:76) and/or mouse/human orthologs/homologs thereof. In some instances, useful TRBV1 regulatory sequences may include sequence upstream of a TRBV1 start site including but not limited to e.g., sequence more than 5 kb or 5 kb or less, 4 kb or less, 3 kb or less, 2 kb or less, or 1 kb or less upstream from a TRBV1 start site, including but not limited to e.g., the start site of UniGene ID Mm.333009.

Useful regulatory sequences include THY1 (also known as CD antigen CD90) regulatory sequences (e.g., a THY1 promoter), including those regulating the expression of a gene encoding RefSeq NP_001298089.1 (SEQ ID NO:36) (Uniprot ID P04216, SEQ ID NO:77) and/or mouse/human orthologs/homologs thereof. In some instances, useful THY1 regulatory sequences may include sequence upstream of a THY1 start site including but not limited to e.g., sequence more than 5 kb or 5 kb or less, 4 kb or less, 3 kb or less, 2 kb or less, or 1 kb or less upstream from a THY1 start site, including but not limited to e.g., the start site of UniGene ID Hs.644697.

Useful regulatory sequences include GM288 (also known as Ccnb1ip1, Hei10 and Mei4) regulatory sequences (e.g., a GM288 promoter), including those regulating the expression of a gene encoding RefSeq NP_001104589.1 (SEQ ID NO:37) (Uniprot ID D3Z3K2, SEQ ID NO:78) and/or mouse/human orthologs/homologs thereof. In some instances, useful GM288 regulatory sequences may include sequence upstream of a GM288 start site including but not limited to e.g., sequence more than 5 kb or 5 kb or less, 4 kb or less, 3 kb or less, 2 kb or less, or 1 kb or less upstream from a GM288 start site, including but not limited to e.g., the start site of UniGene ID Mm.343880.

Useful regulatory sequences include DPP4 (also known as ADCP2 and CD26) regulatory sequences (e.g., a DPP4 promoter), including those regulating the expression of a gene encoding RefSeq NP_001926.2 (SEQ ID NO:38) (Uniprot ID P27487, SEQ ID NO:79) and/or mouse/human orthologs/homologs thereof. In some instances, useful DPP4 regulatory sequences may include sequence upstream of a DPP4 start site including but not limited to e.g., sequence more than 5 kb or 5 kb or less, 4 kb or less, 3 kb or less, 2 kb or less, or 1 kb or less upstream from a DPP4 start site, including but not limited to e.g., the start site of UniGene ID Hs.368912.

Useful regulatory sequences include CTSE (also known as Cathepsin E) regulatory sequences (e.g., a CTSE promoter), including those regulating the expression of a gene encoding RefSeq NP_001304260.1 (SEQ ID NO:39) (Uniprot ID P14091, SEQ ID NO:80) and/or mouse/human orthologs/homologs thereof. In some instances, useful CTSE regulatory sequences may include sequence upstream of a CTSE start site including but not limited to e.g., sequence more than 5 kb or 5 kb or less, 4 kb or less, 3 kb or less, 2 kb or less, or 1 kb or less upstream from a CTSE start site, including but not limited to e.g., the start site of UniGene ID Hs.644082.

Useful regulatory sequences include CD3G (also known as T3G) regulatory sequences (e.g., a CD3G promoter), including those regulating the expression of a gene encoding RefSeq NP_000064.1 (SEQ ID NO:40) (Uniprot ID P09693, SEQ ID NO:81) and/or mouse/human orthologs/homologs thereof. In some instances, useful CD3G regulatory sequences may include sequence upstream of a CD3G start site including but not limited to e.g., sequence more than 5 kb or 5 kb or less, 4 kb or less, 3 kb or less, 2 kb or less, or 1 kb or less upstream from a CD3G start site, including but not limited to e.g., the start site of UniGene ID Hs.2259.

Useful regulatory sequences include IGH-6 (also known as Ig mu chain C region) regulatory sequences (e.g., a IGH-6 promoter), including those regulating the expression of a gene encoding Uniprot ID P01872 (SEQ ID NO:82) and/or mouse/human orthologs/homologs thereof. In some instances, useful IGH-6 regulatory sequences may include sequence upstream of a IGH-6 start site including but not limited to e.g., sequence more than 5 kb or 5 kb or less, 4 kb or less, 3 kb or less, 2 kb or less, or 1 kb or less upstream from a IGH-6 start site, including but not limited to e.g., the start site of UniGene ID Mm.304472.

Useful regulatory sequences include IGH-6 (also known as Ig-region protein) regulatory sequences (e.g., a IGH-6 promoter), including those regulating the expression of a gene encoding Uniprot ID P04219 (SEQ ID NO:83) and/or mouse/human orthologs/homologs thereof. In some instances, useful IGH-6 regulatory sequences may include sequence upstream of a IGH-6 start site including but not limited to e.g., sequence more than 5 kb or 5 kb or less, 4 kb or less, 3 kb or less, 2 kb or less, or 1 kb or less upstream from a IGH-6 start site, including but not limited to e.g., the start site of UniGene ID Mm.304472.

Any convenient cell type may be employed in the subject methods including cancer cell types, including but not limited to e.g., one or more cancer cell types selected from the following cancers: Acute Lymphoblastic Leukemia (ALL), Acute Myeloid Leukemia (AML), Adrenocortical Carcinoma, AIDS-Related Cancers (e.g., Kaposi Sarcoma, Lymphoma, etc.), Anal Cancer, Appendix Cancer, Astrocytomas, Atypical Teratoid/Rhabdoid Tumor, Basal Cell Carcinoma, Bile Duct Cancer (Extrahepatic), Bladder Cancer, Bone Cancer (e.g., Ewing Sarcoma, Osteosarcoma and Malignant Fibrous Histiocytoma, etc.), Brain Stem Glioma, Brain Tumors (e.g., Astrocytomas, Central Nervous System Embryonal Tumors, Central Nervous System Germ Cell Tumors, Craniopharyngioma, Ependymoma, etc.), Breast Cancer (e.g., female breast cancer, male breast cancer, childhood breast cancer, etc.), Bronchial Tumors, Burkitt Lymphoma, Carcinoid Tumor (e.g., Childhood, Gastrointestinal, etc.), Carcinoma of Unknown Primary, Cardiac (Heart) Tumors, Central Nervous System (e.g., Atypical Teratoid/Rhabdoid Tumor, Embryonal Tumors, Germ Cell Tumor, Lymphoma, etc.), Cervical Cancer, Childhood Cancers, Chordoma, Chronic Lymphocytic Leukemia (CLL), Chronic Myelogenous Leukemia (CML), Chronic Myeloproliferative Neoplasms, Colon Cancer, Colorectal Cancer, Craniopharyngioma, Cutaneous T-Cell Lymphoma, Duct (e.g., Bile Duct, Extrahepatic, etc.), Ductal Carcinoma In Situ (DCIS), Embryonal Tumors, Endometrial Cancer, Ependymoma, Esophageal Cancer, Esthesioneuroblastoma, Ewing Sarcoma, Extracranial Germ Cell Tumor, Extragonadal Germ Cell Tumor, Extrahepatic Bile Duct Cancer, Eye Cancer (e.g., Intraocular Melanoma, Retinoblastoma, etc.), Fibrous Histiocytoma of Bone (e.g., Malignant, Osteosarcoma, ect.), Gallbladder Cancer, Gastric (Stomach) Cancer, Gastrointestinal Carcinoid Tumor, Gastrointestinal Stromal Tumors (GIST), Germ Cell Tumor (e.g., Extracranial, Extragonadal, Ovarian, Testicular, etc.), Gestational Trophoblastic Disease, Glioma, Hairy Cell Leukemia, Head and Neck Cancer, Heart Cancer, Hepatocellular (Liver) Cancer, Histiocytosis (e.g., Langerhans Cell, etc.), Hodgkin Lymphoma, Hypopharyngeal Cancer, Intraocular Melanoma, Islet Cell Tumors (e.g., Pancreatic Neuroendocrine Tumors, etc.), Kaposi Sarcoma, Kidney Cancer (e.g., Renal Cell, Wilms Tumor, Childhood Kidney Tumors, etc.), Langerhans Cell Histiocytosis, Laryngeal Cancer, Leukemia (e.g., Acute Lymphoblastic (ALL), Acute Myeloid (AML), Chronic Lymphocytic (CLL), Chronic Myelogenous (CML), Hairy Cell, etc.), Lip and Oral Cavity Cancer, Liver Cancer (Primary), Lobular Carcinoma In Situ (LCIS), Lung Cancer (e.g., Non-Small Cell, Small Cell, etc.), Lymphoma (e.g., AIDS-Related, Burkitt, Cutaneous T-Cell, Hodgkin, Non-Hodgkin, Primary Central Nervous System (CNS), etc.), Macroglobulinemia (e.g., Waldenström, etc.), Male Breast Cancer, Malignant Fibrous Histiocytoma of Bone and Osteosarcoma, Melanoma, Merkel Cell Carcinoma, Mesothelioma, Metastatic Squamous Neck Cancer with Occult Primary, Midline Tract Carcinoma Involving NUT Gene, Mouth Cancer, Multiple Endocrine Neoplasia Syndromes, Multiple Myeloma/Plasma Cell Neoplasm, Mycosis Fungoides, Myelodysplastic Syndromes, Myelodysplastic/Myeloproliferative Neoplasms, Myelogenous Leukemia (e.g., Chronic (CML), etc.), Myeloid Leukemia (e.g., Acute (AML), etc.), Myeloproliferative Neoplasms (e.g., Chronic, etc.), Nasal Cavity and Paranasal Sinus Cancer, Nasopharyngeal Cancer, Neuroblastoma, Non-Hodgkin Lymphoma, Non-Small Cell Lung Cancer, Oral Cancer, Oral Cavity Cancer (e.g., Lip, etc.), Oropharyngeal Cancer, Osteosarcoma and Malignant Fibrous Histiocytoma of Bone, Ovarian Cancer (e.g., Epithelial, Germ Cell Tumor, Low Malignant Potential Tumor, etc.), Pancreatic Cancer, Pancreatic Neuroendocrine Tumors (Islet Cell Tumors), Papillomatosis, Paraganglioma, Paranasal Sinus and Nasal Cavity Cancer, Parathyroid Cancer, Penile Cancer, Pharyngeal Cancer, Pheochromocytoma, Pituitary Tumor, Pleuropulmonary Blastoma, Primary Central Nervous System (CNS) Lymphoma, Prostate Cancer, Rectal Cancer, Renal Cell (Kidney) Cancer, Renal Pelvis and Ureter, Transitional Cell Cancer, Retinoblastoma, Rhabdomyosarcoma, Salivary Gland Cancer, Sarcoma (e.g., Ewing, Kaposi, Osteosarcoma, Rhabdomyosarcoma, Soft Tissue, Uterine, etc.), Sézary Syndrome, Skin Cancer (e.g., Childhood, Melanoma, Merkel Cell Carcinoma, Nonmelanoma, etc.), Small Cell Lung Cancer, Small Intestine Cancer, Soft Tissue Sarcoma, Squamous Cell Carcinoma, Squamous Neck Cancer (e.g., with Occult Primary, Metastatic, etc.), Stomach (Gastric) Cancer, T-Cell Lymphoma, Testicular Cancer, Throat Cancer, Thymoma and Thymic Carcinoma, Thyroid Cancer, Transitional Cell Cancer of the Renal Pelvis and Ureter, Ureter and Renal Pelvis Cancer, Urethral Cancer, Uterine Cancer (e.g., Endometrial, etc.), Uterine Sarcoma, Vaginal Cancer, Vulvar Cancer, Waldenström Macroglobulinemia, Wilms Tumor, and the like.

One or more changes in a MYC-driven cancer cell (e.g., containing a MYC reporter) contacted with a candidate agent may be compared to an appropriate control. In some instances, an appropriate control may include a comparable or the same MYC-driven cancer cell not contacted with the candidate agent. Other controls may be employed including e.g., cells where MYC expression is induced (e.g., enhanced or “ON”) or repressed (e.g., inhibited or “OFF”). A change, or one or more changes, in expression may be qualitatively or quantitatively assessed. For example, in some instances, a change in expression may be measured quantitatively e.g., through two or more measurements made over time (or numerous real-time measurements) of a fluorescent signal detected from a fluorescent reporter. Measurements may be performed on individual cells, across a population of cells, and/or combinations thereof.

The subject methods may further include additional assessments of the subject cells including e.g., viability assessments, cell morphology assessments (e.g., cell size, cell shape, nuclear size/shape, cytoplasm size/shape, etc.), anti-proliferation assessments (e.g., a luminescent cell viability assay), MYC target gene expression assessments (e.g., expression of any convenient MYC target gene (e.g., apurinic/apyrimidinic endodeoxyribonuclease 1 (APEX1)), and the like.

MYC target genes useful in MYC target gene expression assessments include, as summarized above, APEX1 (also known as APE, APX, APE1, APEN, APEX, HAP1, and REF1). In humans, APEX1 is present on chromosome 14 at 14q11.2 (GenBank Gene ID: 328) and encodes the major apurinic/apyrimidinic (AP) endonuclease in human cells. Human APEX1 includes the following amino acid RefSeq sequences (encoded by the corresponding RefSeq nucleotide sequences provided): NP_001231178.1 (SEQ ID NO:84) (NM_001244249.1, SEQ ID NO:85), NP_001632.2 (SEQ ID NO:86) (NM_001641.3, SEQ ID NO:87), NP_542379.1 (SEQ ID NO:88) (NM_080648.2, SEQ ID NO:89), and NP_542380.1 (SEQ ID NO:90) (NM_080649.2, SEQ ID NO:91). In mouse, APEX1 is present on chromosome 14 at 14 26.3 cM (GenBank Gene ID: 11792) and is also known as APE, Apex, HAP1, and Ref-1. Mouse APEX1 includes the following amino acid RefSeq sequence (encoded by the corresponding RefSeq nucleotide sequence): NP_033817.1 (SEQ ID NO:92) (NM_009687.2, SEQ ID NO:93).

In some instances, the subject methods may include regulating (e.g., inducing (i.e., activating) or repressing (i.e., inactivating)) MYC expression in an experimental and/or a control group of cells. As such, in some instances, cells employed in the method (e.g., MYC-driven cancer cells) may conditionally express MYC. The subject methods may include contacting the cells with an agent that induces the conditional expression of MYC or that represses the conditional expression of MYC. The subject methods may further include comparing the detected change in the expression of the at least one detectable reporter of a MYC-reporter expression vector in a MYC-driven cancer cell to conditional MYC inactivation of the of MYC-driven cancer cell.

As summarized above, the instant methods include screening candidate agents for activity in a subject screening assay. Any useful inhibitor or activator may be employed as a candidate agent in the subject methods. Non-limiting examples of useful agents include but are not limited to e.g., non-peptide small molecule antagonists/agonists, peptide antagonists/agonists, interfering RNAs (e.g., siRNA, shRNA, etc.), antibodies (e.g., neutralizing antibodies, function blocking antibodies, etc.), aptamers, and the like. Accordingly, as non-limiting examples, in some instances, useful agents may include a non-peptide small molecule antagonists of MYC of a protein involved in MYC signaling, a peptide antagonist of MYC of a protein involved in MYC signaling, an interfering RNA targeting an RNA expressed from a MYC gene or a MYC target gene, an anti-MYC antibody (e.g., an antibody that specifically binds to MYC or otherwise prevents MYC signaling), an anti-MYC aptamer or an aptamers that otherwise prevents MYC signaling, and the like. In some instances, the effectiveness of an agent may be confirmed using an in vitro or in vivo assay, including e.g., where the effectiveness of the agent is compared to an appropriate control or standard, e.g., a conventional therapy for the condition, etc. In some instances, a library of agents may be screened, including e.g., a non-peptide small molecule library, a peptide library, an interfering RNA (e.g., siRNA, shRNA, etc.) library, an antibody (e.g., neutralizing antibodies, function blocking antibodies, etc.) library, an aptamer library, libraries containing a combination of agents thereof, and the like.

Candidate agents identified as useful in treating a MYC driven neoplasia (e.g., agents identified as repressors or inhibitors of MYC) may be employed to treat an individual with the neoplasia. As used herein “neoplasia” includes any form of abnormal new tissue formation; and the like. In some cases, the individual has recently undergone treatment for neoplasia (e.g., cancer, a tumor, etc.) and are therefore at risk for recurrence. In some instances, the individual has not recently or previously undergone treatment for a neoplasia (e.g., cancer, a tumor, etc.) but has been newly diagnosed with a neoplasia. Any and all neoplasia are suitable neoplasia to be treated by the subject methods e.g., utilizing a subject MYC repressor identified according to a screening method described herein.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., room temperature (RT); seconds (s or sec); minutes (m or min); hours (h or hr); days (d); weeks (wk or wks); picoliters (pl), nanoliters (nl); microliters (ul); milliliters (ml); liters (L); nanograms (ng); micrograms (ug); milligrams (mg); grams ((g), in the context of mass); kilograms (kg); equivalents of the force of gravity ((g), in the context of centrifugation); nanomolar (nM); micromolar (uM), millimolar (mM); molar (M); amino acids (aa); kilobases (kb); base pairs (bp); nucleotides (nt); intramuscular (i.m.); intraperitoneal (i.p.); subcutaneous (s.c.); and the like.

### Example 1: Identification of Compounds that Phenocopy Myc Inactivation

Conditional transgenic mouse models to demonstrate that cancers are MYC oncogene addicted have been developed (Li, Y., S. C. Casey, and D. W. Felsher, Inactivation of MYC reverses tumorigenesis. J Intern Med, 2014. 276(1): p. 52-60; Gabay, M., Y. Li, and D. W. Felsher, MYC activation is a hallmark of cancer initiation and maintenance. Cold Spring Harb Perspect Med, 2014. 4(6)). Importantly, in such models, endogenous mouse Myc is not influenced but when the expression of transgenic human MYC is suppressed, this is sufficient to induce sustained tumor regression.

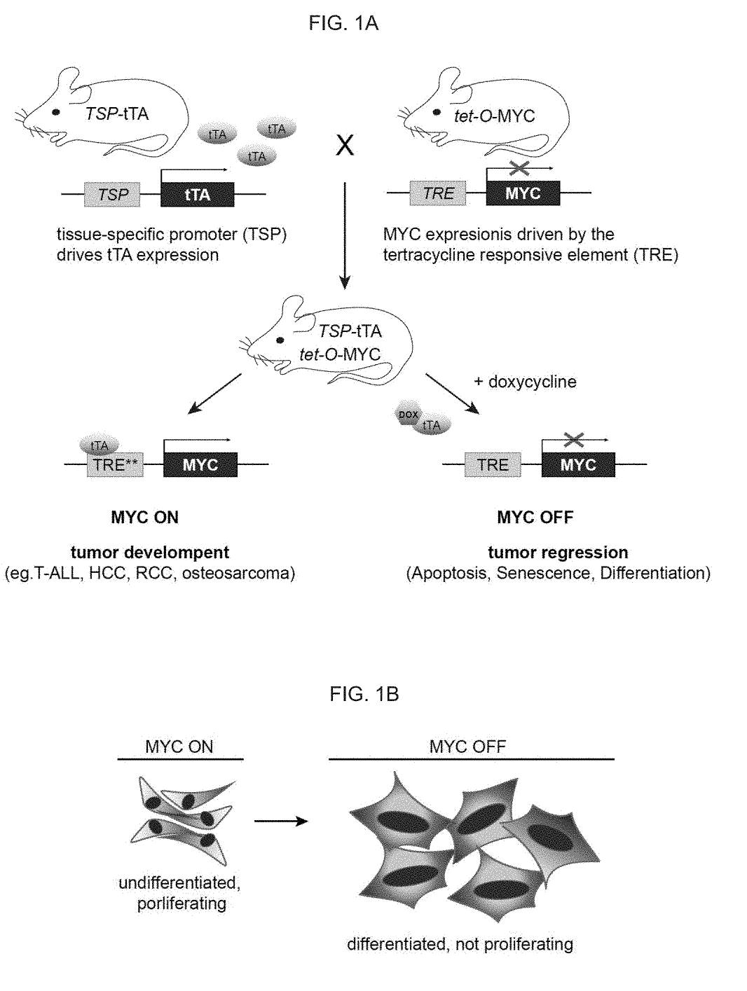

MYC has been widely considered “undruggable”. However, otherwise often untreatable cancers, such as HCC, may be curable with targeted MYC inactivation. MYC is one of the most commonly activated oncogenes of human HCC (Chan, K. L., X. Y. Guan, and I. O. Ng, High-throughput tissue microarray analysis of c-myc activation in chronic liver diseases and hepatocellular carcinoma. Hum Pathol, 2004. 35(11): p. 1324-31; Kaposi-Novak, P., et al., Central role of c-Myc during malignant conversion in human hepatocarcinogenesis. Cancer Res, 2009. 69(7): p. 2775-82; Kawate, S., et al., Amplification of c-myc in hepatocellular carcinoma: correlation with clinicopathologic features, proliferative activity and p53 overexpression. Oncology, 1999. 57(2): p. 157-63. Peng, S. Y., P. L. Lai, and H. C. Hsu, Amplification of the c-myc gene in human hepatocellular carcinoma: biologic significance. J Formos Med Assoc, 1993. 92(10): p. 866-70). MYC is either directly activated through genomic amplification or epistatically activated through other oncogenic events. A conditional transgenic mouse model of HCC has been developed by the inventors of the present application to gain new mechanistic insights, and identify potential new therapeutics for HCC (FIG. 1A-1B).

In this example, identified candidate MYC inhibitors have been further evaluated through examination in vitro using tumor-derived cell lines from MYC-induced mouse models. The generation of the employed transgenic mouse model of MYC-induced cancer is schematically depicted in FIG. 1A. In the produced tissue specific promoter—tetracycline transactivator tet-O operator MYC (TSP-tTA tet-O-MYC) mice the tetracycline transactivator (tTA) is expressed under a tissue-specific promoter and MYC is expressed under the control of the tetracycline-responsive element (TRE). Thus, MYC expression in the target tissue can be regulated by administration of doxycycline (DOX) such that in the absence of DOX, MYC is expressed (MYC ON) and mice develop tissue-specific tumors. Conversely, in the presence of DOX, MYC expression is inactivated (MYC OFF) and tumors regress. Cell lines are derived from the produced mice allowing for investigation and manipulation of MYC expression in in vitro culture (FIG. 1B). “MYC ON” in vitro cultured cells generally display an undifferentiated/proliferating phenotype whereas “MYC OFF” cells generally display a differentiated/non-proliferating phenotype. Such, MYC-induced cancer models, i.e., in vitro, in vivo or both, may employed in the screening of candidate agents.

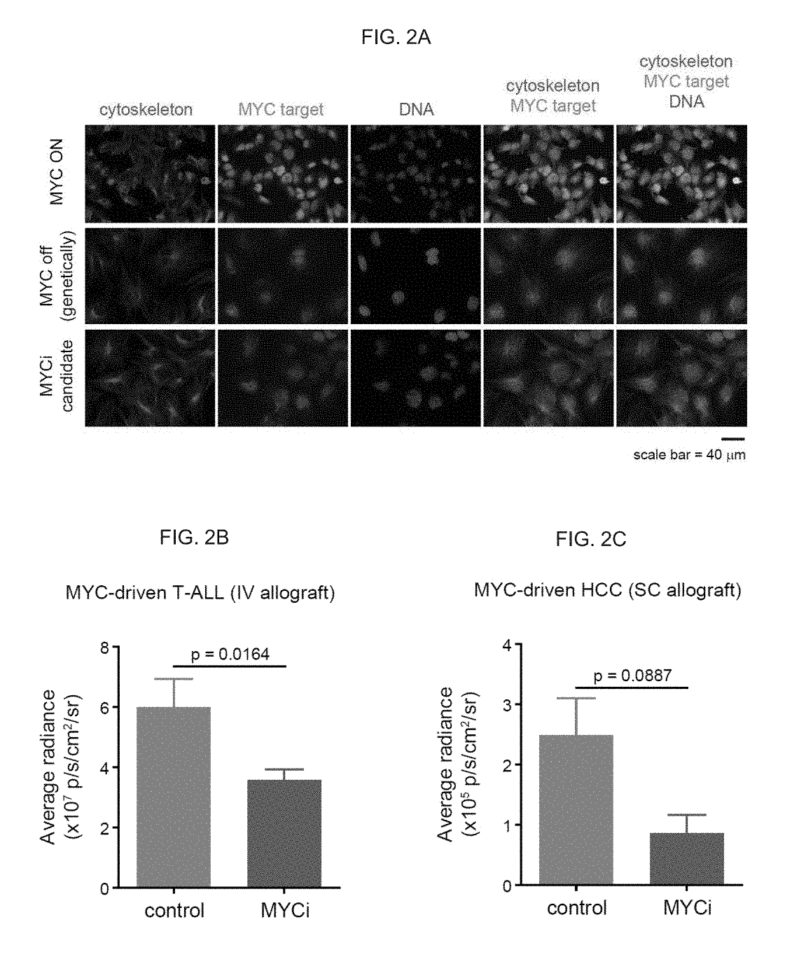

Candidate molecules were assessed for anti-proliferative properties in tumor-derived cell lines from a mouse model of MYC-driven T-ALL (Felsher, D. W. and J. M. Bishop, Reversible tumorigenesis by MYC in hematopoietic lineages. Mol Cell, 1999. 4(2): p. 199-207). In one example, sensitivity to novel MYC-inhibitor candidate agents (also referred to generally as MYC-inhibitors or “MYCi” candidate agents) was measured by a luminescent cell viability assay in vitro. Compounds with the lowest IC50 values were then analyzed for the ability to induce differentiation, using a MYC-driven OS cell line that undergoes terminal differentiation upon MYC inactivation (Jain, M., et al., Sustained loss of a neoplastic phenotype by brief inactivation of MYC. Science, 2002. 297(5578): p. 102-4). Differentiation-associated morphological changes were measured by visualizing cytoskeleton protein (e.g., alpha-tubulin) in combination with changes of expression of a MYC target gene (e.g., APEX1). Both were visualized by immunofluorescence imaging using specific antibodies in a MYC-driven osteosarcoma cell line treated with MYCi candidate or with doxycycline to inactivate MYC expression (FIG. 2A). Accordingly, this approach allows for simultaneous assessment of morphology and MYC activity.

The results showed that cells treated with the candidate MYCi are indistinguishable from cells in which MYC expression was shut off genetically through the use of doxycycline (“MYC off”) (FIG. 2A). Put another way, treatment with the candidate MYCi resulted in differentiation and downregulation of APEX1 expression relative to “MYC ON” controls (FIG. 2A, see “MYCi candidate” as compared to “MYC ON”).

This representative candidate MYCi was further tested in vivo using NGS allograft mouse models bearing MYC-driven luciferase-labeled T-cell acute lymphoblastic leukemia (T-ALL) cells (FIG. 2B) or hepatocellular carcinoma (HCC) (FIG. 2C) cells, using a bioluminescence readout as a measure of tumor burden. In this assay, luciferase-labeled T-ALL cells (4188-LUC) were injected into NOD/SCID/IL-2Rγnull (NSG) mice intravenously (IV). After tumor engraftment, mice were treated IV with either vehicle or candidate MYCi. Mice treated with candidate MYCi showed a significant reduction in tumor burden when compared to vehicle treated mice (FIG. 2B). NSG mice with sub-cutaneous transplants of MYC-driven HCC cells (EC4-LUC) were treated IP with either vehicle or candidate MYCi. Mice treated with candidate MYCi showed a significant reduction in tumor growth compared to vehicle treated mice (FIG. 2C). Thus, the novel small molecule MYCi was identified as a promising candidate for further evaluation as a potential therapeutic for targeting MYC for the treatment of human cancer.

In addition, to identify compounds that induce a similar phenotype as genetic MYC inactivation in a high-throughput approach, a previously developed immunofluorescence staining-based method that can identify molecules that phenocopy conditional MYC inactivation resulting in decreased target gene expression and induction of cellular differentiation was adapted. This adapted method is compatible with high-content imaging, so that it can be used for high-throughput screening approaches.

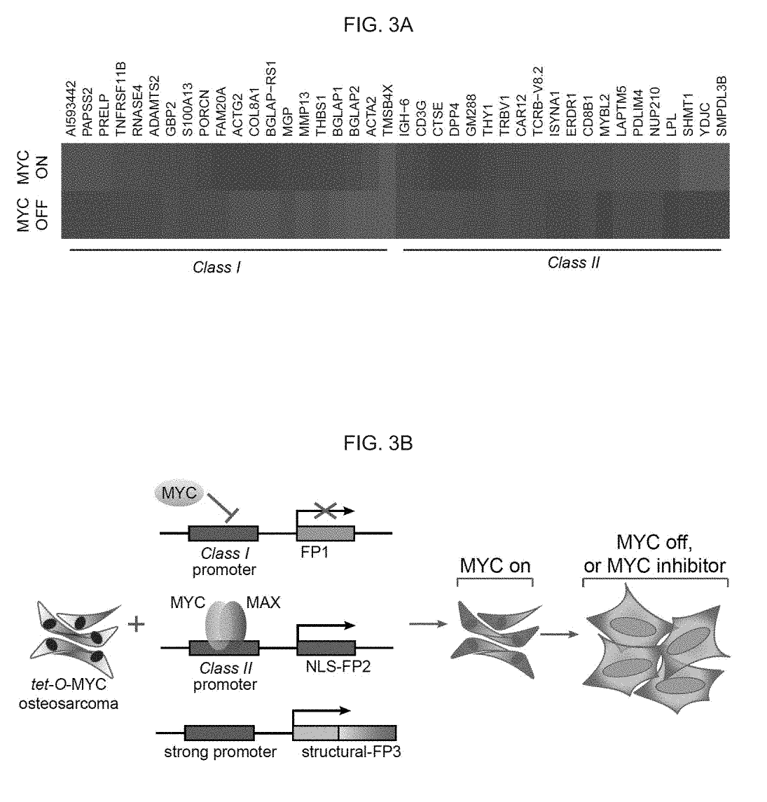

In the instant example, a tumor-derived MYC-induced OS cell line was genetically engineered to express fluorescent reporters that are under the control of MYC responsive promoters. Expression of fluorescently tagged structural proteins was also employed to enable visualization of morphological changes associated with cellular differentiation (FIG. 3A-3B). A reporter cell line, described in more detail below, was specifically developed for this approach. This cell line has the unique advantage that upon MYC inactivation it undergoes differentiation and cellular senescence, but not apoptosis, as has been observed in many other tumors. In this cell line, the complete suppression of MYC expression by addition of doxycycline serves as positive control. Thus, this adapted reporter cell line allows for identification of MYC inhibitors (leading to differentiation) but also discriminates unspecific toxicity (leading to cell death).

To identify MYC regulated promoters for use in the described cell line, changes in gene expression upon MYC inactivation (i.e., MYC shut off) in MYC-driven OS cells were examined. Forty genes/proteins with highest expression changes were identified and classified as “Class I” (i.e., proteins driven by Class I promoters downregulated by MYC) and “Class II” (i.e., proteins driven by Class 2 promoters upregulated by MYC) (FIG. 3A). In this example and some embodiments, an Ebox tandem repeat promoter may serve as a Class II promoter.

To develop a reporter cell line that reports on MYC expression, a MYC-driven OS cell line was genetically engineered to express fluorescent reporters (referred to as fluorescent protein 1 (FP1) and fluorescent protein 2 (FP2)) under the control of a Class I promoter (e.g., the MMP13 promoter) and a Class II promoter (e.g., the NUP210 promoter). One or both fluorescent reporters (e.g, GFP and E2-Crimson) may are targeted to the nucleus, which simplifies image segmentation and quantitative image analysis. In the example depicted (FIG. 3B) FP2 was targeted to the nucleus via a nuclear localization sequence (NLS). In addition, cell morphology was visualized by expression of fluorescently tagged structural protein (e.g., Actin or a microtubule protein fused to a third fluorescent protein (FP3)), e.g., under the control of a strong promoter, such as a strong constitutive promoter. Then, the engineered reporter cell line (FIG. 3B) was used for screening MYC inhibitor candidates by high-content imaging in a 384-well format. High-content imaging was performed using a high-content imaging system (ImageXpress Micro, Molecular Devices).

This newly developed high-throughput screening approach allows for the efficient identification of MYC inhibitors. With respect to the example depicted in FIG. 3B, molecules that specifically inhibit MYC function lead to the induction of FP1 expression, the loss of FP2 expression, and an increase in structural-protein-FP3-positive cytoplasmic area. Thus, this system allows for the identification of a unique phenotype that recapitulates suppression of MYC expression in a MYC-driven cancer cell line. In addition, specific inhibitors do not interfere with the Tet-system (no induction of luciferase expression in the Tet-ON reporter), and also show efficacy in tumor cell lines with high endogenous MYC levels or constitutive MYC expression. Compounds that meet the above criteria can be further evaluated for in vivo testing, including e.g., further testing in MYC-induced in vivo cancer models such as the MYC-induced in vivo models (e.g., the MYC-induced HCC mouse model) described above. Compounds that do not meet either of these criteria are identified as non-specific or not efficacious.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

1. A MYC-reporter expression vector comprising a nucleotide sequence encoding a detectable reporter operably linked to a MYC-responsive element comprising a regulatory sequence of a MYC-responsive gene selected from the group consisting of: expressed sequence AI593442 (AI593442); 3′-phosphoadenosine 5′-phosphosulfate synthase 2 (PAPSS2); proline and arginine rich end leucine rich repeat protein (PRELP); TNF receptor superfamily member 11b (TNFRSF11B); ribonuclease A family member 4 (RNASE4); ADAM metallopeptidase with thrombospondin type 1 motif 2 (ADAMTS2); guanylate binding protein 2 (GBP2); S100 calcium binding protein A13 (S100A13); porcupine homolog (PORCN); FAM20A, golgi associated secretory pathway pseudokinase (FAM20A); actin, gamma 2, smooth muscle, enteric (ACTG2); collagen type VIII alpha 1 chain (COL8A1); bone gamma-carboxyglutamate protein 3 (BGLAP-RS1); matrix Gla protein (MGP); matrix metallopeptidase 13 (MMP13); thrombospondin 1 (THBS1); bone gamma carboxyglutamate protein (BGLAP1); bone gamma-carboxyglutamate protein 2 (BGLAP2); actin, alpha 2, smooth muscle, aorta (ACTA2); thymosin beta 4, X-linked (TMSB4X); sphingomyelin phosphodiesterase acid like 3B (SMPDL3B); YdjC homolog (YDJC); serine hydroxymethyltransferase 1 (SHMT1); lipoprotein lipase (LPL); nucleoporin 210 (NUP210); PDZ and LIM domain 4 (PDLIM4); lysosomal protein transmembrane 5 (LAPTM5); MYB proto-oncogene like 2 (MYBL2); CD8b molecule (CD8B1); erythroid differentiation regulator 1 (ERDR1); inositol-3-phosphate synthase 1 (ISYNA1); T cell receptor beta, variable 13-2 (TCRB-V8.2); carbonic anhydrase 12 (CAR12); T cell receptor beta variable 1 (TRBV1); Thy-1 cell surface antigen (THY1); cyclin B1 interacting protein 1 (GM288); dipeptidyl peptidase 4 (DPP4); cathepsin E (CTSE); CD3g molecule (CD3G) and immunoglobulin heavy constant mu (IGH-6).

2. The MYC-reporter expression vector according to claim 1, wherein the MYC-responsive gene is NUP210 or TRBV1.

3. The MYC-reporter expression vector according to claim 1, wherein the expression vector comprises two or more nucleotide sequences encoding a detectable reporter each operably linked to a MYC-responsive element comprising a regulatory sequence of a MYC-responsive gene selected from the group.

4. (canceled)

5. The MYC-reporter expression vector according to claim 1, wherein the detectable reporter is a fluorescent protein and the fluorescent protein is a destabilized fluorescent protein.

6. The MYC-reporter expression vector according to claim 1, wherein the expression vector further comprises a nucleotide sequence encoding a tagged structural protein.

7-8. (canceled)

9. The MYC-reporter expression vector according to claim 1, wherein the expression vector comprises a tandem Ebox element operably linked to a nucleotide sequence encoding a detectable reporter.

10. A cancer cell comprising the MYC-reporter expression vector according to claim 1.

11. The cancer cell according to claim 10, wherein the cancer cell line is a hepatocellular carcinoma cell, a T-cell acute lymphoblastic leukemia cell, an osteosarcoma cell, a renal cell carcinoma cell or a lung cancer cell.

12. The cancer cell according to claim 10, wherein the cancer cell comprises an inducible MYC or a constitutive MYC.

13-14. (canceled)

15. A transgenic non-human animal comprising the cancer cell according to claim 10.

16. (canceled)

17. A multi-MYC-reporter expression system, the system comprising:
a nucleotide sequence encoding a first detectable reporter operably linked to a regulatory sequence that is upregulated by MYC; and
a nucleotide sequence encoding a second detectable reporter operably linked to regulatory sequence that is downregulated by MYC.

18. The multi-MYC-reporter expression system according to claim 17, wherein the regulatory sequence that is downregulated by MYC is selected from the group consisting of: expressed sequence AI593442 (AI593442); 3′-phosphoadenosine 5′-phosphosulfate synthase 2 (PAPSS2); proline and arginine rich end leucine rich repeat protein (PRELP); TNF receptor superfamily member 11b (TNFRSF11B); ribonuclease A family member 4 (RNASE4); ADAM metallopeptidase with thrombospondin type 1 motif 2 (ADAMTS2); guanylate binding protein 2 (GBP2); S100 calcium binding protein A13 (S100A13); porcupine homolog (PORCN); FAM20A, golgi associated secretory pathway pseudokinase (FAM20A); actin, gamma 2, smooth muscle, enteric (ACTG2); collagen type VIII alpha 1 chain (COL8A1); bone gamma-carboxyglutamate protein 3 (BGLAP-RS1); matrix Gla protein (MGP); matrix metallopeptidase 13 (MMP13); thrombospondin 1 (THBS1); bone gamma carboxyglutamate protein (BGLAP1); bone gamma-carboxyglutamate protein 2 (BGLAP2); actin, alpha 2, smooth muscle, aorta (ACTA2) and thymosin beta 4, X-linked (TMSB4X).

19. (canceled)

20. The multi-MYC-reporter expression system according to claim 17, wherein the regulatory sequence that is upregulated by MYC is selected from the group consisting of: sphingomyelin phosphodiesterase acid like 3B (SMPDL3B); YdjC homolog (YDJC); serine hydroxymethyltransferase 1 (SHMT1); lipoprotein lipase (LPL); nucleoporin 210 (NUP210); PDZ and LIM domain 4 (PDLIM4); lysosomal protein transmembrane 5 (LAPTM5); MYB proto-oncogene like 2 (MYBL2); CD8b molecule (CD8B1); erythroid differentiation regulator 1 (ERDR1); inositol-3-phosphate synthase 1 (ISYNA1); T cell receptor beta, variable 13-2 (TCRB-V8.2); carbonic anhydrase 12 (CAR12); T cell receptor beta variable 1 (TRBV1); Thy-1 cell surface antigen (THY1); cyclin B1 interacting protein 1 (GM288); dipeptidyl peptidase 4 (DPP4); cathepsin E (CTSE); CD3g molecule (CD3G) and immunoglobulin heavy constant mu (IGH-6).

21-30. (canceled)

31. A cancer cell comprising the multi-MYC-reporter expression system according to claim 17.

32-35. (canceled)

36. A transgenic non-human animal comprising the cancer cell according to claim 31.

37. (canceled)

38. A method of screening a candidate agent for MYC repression, the method comprising:
contacting a MYC-driven cancer cell comprising a MYC-reporter expression vector with the candidate agent; and
detecting a change in the expression of at least one detectable reporter of the MYC-reporter expression vector in the MYC-driven cancer cell during or following the contacting as compared to a control MYC-driven cancer cell not contacted with the candidate agent.

39. The method according to claim 38, wherein the method further comprises an anti-proliferation assessment of the candidate agent.

40. (canceled)

41. The method according to claim 38, wherein the contacting is performed in vitro or in vivo.

42-43. (canceled)

44. The method according claim 38, wherein the MYC-driven cancer cell is a human cancer cell with elevated MYC expression.

45-46. (canceled)

47. The method according to claim 38, wherein the method further comprises detecting a morphological change in the MYC driven cancer cell during or following the contacting as compared to the control MYC-driven cancer cell not contacted with the candidate agent.

48. The method according to claim 47, wherein the morphological change comprises a change in cytoplasmic area of the cancer cell.

49. The method according to claim 38, wherein the method further comprises quantifying the expression of a MYC target gene.

50. The method according to claim 49, wherein the MYC target gene is apurinic/apyrimidinic endodeoxyribonuclease 1 (APEX1).

51. (canceled)

52. The method according to claim 38, wherein the method further comprises comparing the detected change in the expression of the at least one detectable reporter of the MYC-reporter expression vector in the MYC-driven cancer cell to conditional MYC inactivation of the of MYC-driven cancer cell.
